(19) United States
(12) Patent Application Publication
(10) Pub. No.: US 2015/0159216 A1
(43) Pub. Date: Jun. 11, 2015

(54) METHODS AND COMPOSITIONS RELATED TO THE SMCHD1 GENE

(71) Applicants: FRED HUTCHINSON CANCER RESEARCH CENTER, Seattle, WA (US); LEIDEN UNIVERSITY MEDICAL CENTER, Leiden (NL); UNIVERSITY OF ROCHESTER MEDICAL CENTER, Rochester, NY (US); UNIVERSITY OF WASHINGTON THROUGH ITS CENTER FOR COMMERCIALIZATION, Seattle, WA (US)

(72) Inventors: Stephen Tapscott, Seattle, WA (US); Daniel G. Miller, Seattle, WA (US); Silvere M. van der Maarel, Leiden (NL); Rabi Tawil, Rochester, NY (US)

(21) Appl. No.: 14/409,192

(22) PCT Filed: Jun. 18, 2013
(86) PCT No.: PCT/US2013/046332

§ 371 Date: Dec. 18, 2014

Related U.S. Application Data
(60) Provisional application No. 61/661,236, filed on Jun. 18, 2012.

(51) Int. Cl. C12Q 1/68; G01N 33/68; C12N 15/113; C07K 14/47

(52) U.S. Cl. CPC C12Q 1/6883; C07K 14/4707; G01N 33/6887; C12N 15/113; C12Q 2600/156; C12Q 2600/158; C12Q 2600/112; G01N 2333/4712; G01N 2800/10; C12N 2320/30; C12N 2310/14

(57) ABSTRACT

The present invention relates generally to the field of molecular biology and genetics. More particularly, it concerns methods and compositions for detecting, diagnosing, and/or treating facioscapulohumeral dystrophy (FSHD2).

### CROSS-REFERENCE TO RELATED APPLICATION

This application claims the benefit of priority to U.S. Provisional Application Ser. No. 61/661,236, filed Jun. 18, 2012, hereby incorporated by reference in its entirety.

This invention was made with government support under P01NS06939 and 5RC2HG005608-02 awarded by the National Institutes of Health. The government has certain rights in the invention.

### BACKGROUND

I. Field of the Invention

The present invention relates generally to the field of molecular biology and genetics. More particularly, it concerns methods and compositions for detecting, diagnosing, and/or treating Facioscapulohumeral dystrophy (FSHD), as well as other diseases subject to epigenetic regulation.

II. Background

Facioscapulohumeral muscular dystrophy (FSHMD, FSHD, or FSH) is a usually autosomal dominant inherited form of muscular dystrophy that initially affects the skeletal muscles of the face, scapula, and upper arms (Lemmers et. al., 2007). FSHD is widely considered as the third most common genetic disease of skeletal muscle. A 2008 analysis of rare diseases listed FSHD as the most prevalent form of muscular dystrophy at 7/100,000 (Orphanet Report Series).

Facioscapulohumeral dystrophy (FSHD) is characterized by chromatin relaxation of the D4Z4 macrosatellite array on chromosome 4 and the variegated expression of the D4Z4-encoded DUX4 gene in skeletal muscle nuclei. The more common form, FSHD1, is caused by a contraction of the D4Z4 array and is inherited as a dominant trait, whereas the genetic determinants and inheritance of D4Z4 array contraction-independent FSHD2 are unclear.

Therefore, there is a need for identifying a novel target, such as an epigenetic modifier of genomic regions, and for developing methods and compositions for detecting, diagnosing and/or treating FSHD disease, and other diseases subject to epigenetic regulation.

### SUMMARY OF THE INVENTION

Some embodiments are partly based on the finding that SMCHD1 (structural maintenance of chromosomes flexible hinge domain containing 1) is identified as an epigenetic modifier of the D4Z4 metastable epiallele, a causal genetic determinant of FSHD2, and a modifier of the severity of FSHD1 and other human diseases subject to epigenetic regulation. It has been found that rare, normally benign variants in SMCHD1 on chromosome 18, including mutations and polymorphisms in the gene SmchD1, reduce SMCHD1 protein levels and segregate with genome-wide D4Z4 CpG hypomethylation in human kindreds. FSHD2 occurs in individuals who inherited both the SMCHD1 variant and a normal-sized D4Z4 array on a chromosome 4 haplotype permissive for DUX4 mRNA polyadenylation. Reducing SMCHD1 levels in skeletal muscle results in contraction-independent variegated expression of DUX4. The identification of mutation and polymorphism of SMCHD1 as a cause for FSHD2 and modifiers of other human diseases and syndromes, including FSHD1, opens the door to diagnostic and therapeutic applications. Accordingly, methods and compositions are provided and described.

Generally, embodiments involve an isolated DNA molecule comprising a non-genomic sequence of human SMCHD1 gene, which comprises a SMCHD1 gene variant that reduces SMCHD1 activity in a cell compared to a cell with a wild-type SMCHD1 sequence. In certain embodiments, a SMCHD1 gene variant is a deletion variant, including both out-of-frame deletion variant and in-frame deletion variant, a splice-site variant or a missense variant. In additional embodiments, a SMCHD1 gene variant comprises g.2697999_{—}26098003del, g.2700705G>C, g.2700743T>C, g.2700875_{—}2700875del, g.2701019A>G, g.2707565C>T, g.2722661G>A, g.2732488_{—}2732492del, g.2739448T>A, g.2743927G>A, g.2762234G>A, or g.2763729T>C variant (Numbering refers to chromosome 18 (GRCh37/hg19 Human Genome Assembly) as it appears on the UCSC genome browser, available on the world wide web at genome.ucsc.edu/cgi-bin/hgGateway). In some embodiments, a SMCHD1 gene variant comprises g.2656098del, g.2667016G>A, g.2667018C>T, g.2667031G>A, g.2674086C>T, g.2674088dup, g.2688624A>G, g.2688659C>G, g.2688659C>G, g.2694681C>T, g.2697047A>G, g.2697047A>G, g.2697970G>A, g.269799_{—}2698003del, g.2700630G>C, g.2700743T>C, g.2700849C>T, g.2700875_{—}2700875del, g.2700919A>G, g.2705677A>G, g.2705691G>T, g.2707565C>T, g.2707565C>T, g.2707643G>A, g.2707804G>C, g.2707849C>T, g.2707901T>C, g.2707923G>A, g.2718233G>T, g.2722603G>A, g.2722661G>A, g.2724958dup, g.2729408G>T, g.2729409T>C, g.2732452_{—}2732453del, g.2732488_{—}2732491del, g.2732488_{—}2732491del, g.2732490_{—}2732494del, g.2732490_{—}2732494del, g.2732494_{—}2732497del, g.2740713_{—}2740715delinsTGC, g.2743740A>G, g.2743740A>G, g.2743927G>A, g.2743927G>A, g.2760707G>A, g.2762125_{—}2762130dup, g.2762234G>A, g.2762234G>A, g.2763729T>C, g.2769710delA, g.2769710delA, g.2777820C>T, g.2784496C>G, g.2784502C>T, or g.2795945A>C.

In certain embodiments, the isolated DNA molecule described herein has or has less than 1000, 800, 500, 250, 100, 50 or 25 nucleotides, including all values derivable there between.

In some embodiments, the isolated DNA molecule described herein is modified with a label. The label may comprise a detectable compound or substance. In some embodiments, the label can be a fluorescent, radioactive, enzymatic, colorimetric, metallic, electrochemical or radioactive label. The label may be coupled to the isolated DNA by an enzymatic method or a chemical method.

Additional embodiments involve an isolated nucleic acid fragment and non-naturally occurring nucleic acid fragments comprising a SMCHD1 gene variant. The presence of the variant in a subject reduces SMCHD1 protein levels in the subject as compared to a subject that does not have the variant or as compared to a subject that carries a wild-type SMCHD1 gene sequence. In certain embodiments, a SMCHD1 gene variant is a deletion variant, including both out-of-frame deletion variants and in-frame deletion variants, a splice-site variant or a missense variant. In additional embodiments, a SMCHD1 gene variant comprises one or more mutations, including, but not limited to g.2697999_{—}26098003del, g.2700705G>C, g.2700743T>C, g.2700875_{—}2700875del, g.2701019A>G, g.2707565C>T, g.2722661G>A, g.2732488_{—}2732492del, g.2739448T>A, g.2743927G>A, g.2762234G>A, or g.2763729T>C. In some embodiments, a SMCHD1 gene variant comprises g.2656098del, g.2667016G>A, g.2667018C>T, g.2667031G>A, g.2674086C>T, g.2674088dup, g.2688624A>G, g.2688659C>G, g.2688659C>G, g.2694681C>T, g.2697047A>G, g.2697047A>G, g.2697970G>A, g.269799_{—}2698003del, g.2700630G>C, g.2700743T>C, g.2700849C>T, g.2700875_{—}2700875del, g.2700919A>G, g.2705677A>G, g.2705691G>T, g.2707565C>T, g.2707565C>T, g.2707643G>A, g.2707804G>C, g.2707849C>T, g.2707901T>C, g.2707923G>A, g.2718233G>T, g.2722603G>A, g.2722661G>A, g.2724958dup, g.2729408G>T, g.2729409T>C, g.2732452_{—}2732453del, g.2732488_{—}2732491del, g.2732488_{—}2732491del, g.2732490_{—}2732494del, g.2732490_{—}2732494del, g.2732494_{—}2732497del, g.2740713_{—}2740715delinsTGC, g.2743740A>G, g.2743740A>G, g.2743927G>A, g.2743927G>A, g.2760707G>A, g.2762125_{—}2762130dup, g.2762234G>A, g.2762234G>A, g.2763729T>C, g.2769710delA, g.2769710delA, g.2777820C>T, g.2784496C>G, g.2784502C>T, or g.2795945A>C.

Certain embodiments provide methods for detecting facioscapulohumeral dystrophy 2 (FSHD2) comprising assaying for the presence of a variant in one or both alleles of a SMCHD1 gene in a sample from a subject. The variant is a mutation or a polymorphism associated with a reduction of SMCHD1 activity compared to a wild-type SMCHD1 gene. In some aspects, the variant is a deletion variant, including both out-of-frame deletion variants and in-frame deletion variants, a splice-site variant or a missense variant. In additional embodiments, the variant comprises one or more mutations, including, but not limited to g.2697999_{—}26098003del, g.2700705G>C, g.2700743T>C, g.2700875_{—}2700875del, g.2701019A>G, g.2707565C>T, g.2722661G>A, g.2732488_{—}2732492del, g.2739448T>A, g.2743927G>A, g.2762234G>A, or g.2763729T>C. In some embodiments, a SMCHD1 gene variant comprises g.2656098del, g.2667016G>A, g.2667018C>T, g.2667031G>A, g.2674086C>T, g.2674088dup, g.2688624A>G, g.2688659C>G, g.2688659C>G, g.2694681C>T, g.2697047A>G, g.2697047A>G, g.2697970G>A, g.269799_{—}2698003del, g.2700630G>C, g.2700743T>C, g.2700849C>T, g.2700875_{—}2700875del, g.2700919A>G, g.2705677A>G, g.2705691G>T, g.2707565C>T, g.2707565C>T, g.2707643G>A, g.2707804G>C, g.2707849C>T, g.2707901T>C, g.2707923G>A, g.2718233G>T, g.2722603G>A, g.2722661G>A, g.2724958dup, g.2729408G>T, g.2729409T>C, g.2732452_{—}2732453del, g.2732488_{—}2732491del, g.2732488_{—}2732491del, g.2732490_{—}2732494del, g.2732490_{—}2732494del, g.2732494_{—}2732497del, g.2740713_{—}2740715delinsTGC, g.2743740A>G, g.2743740A>G, g.2743927G>A, g.2743927G>A, g.2760707G>A, g.2762125_{—}2762130dup, g.2762234G>A, g.2762234G>A, g.2763729T>C, g.2769710delA, g.2769710delA, g.2777820C>T, g.2784496C>G, g.2784502C>T, or g.2795945A>C.

In some aspects, the methods described herein involve detecting the presence of the variant described herein in one or both alleles in the sample from the subject. In further aspects, a variant in both alleles of a SMCHD1 gene is detected. In additional aspects, methods provided herein further comprises identifying the subject having a biomarker indicative of FSHD2.

In some embodiments, methods described herein may comprise any of the following steps described herein: obtaining a sample from a subject; isolating nucleic acid molecules from the sample; isolating genomic DNA from the sample; isolating RNA from the sample; synthesizing DNA complementary (cDNA) to the isolated RNA; amplifying the isolated nucleic acid molecules; sequencing one or both alleles; hybridizing with the isolated nucleic acid molecules with a probe; obtaining the sequence information of SMCHD1 gene on one or both alleles; and comparing the obtained sequence information of SMCHD1 gene with wild type SMCHD1 gene.

In some embodiments, methods may further involve one or more of the following steps regarding nucleic acids prior to and/or concurrent with detecting a SMCHD1 variant: obtaining nucleic acid molecules; obtaining nucleic acid molecules from a biological sample; obtaining a biological sample containing nucleic acids from a subject; isolating nucleic acid molecules; purifying nucleic acid molecules; amplifying nucleic acid molecules; obtaining an array or microarray containing nucleic acids to be analyzed; denaturing nucleic acid molecules; shearing or cutting nucleic acid; denaturing nucleic acid molecules; hybridizing nucleic acid molecules; incubating the nucleic acid molecule with an enzyme; incubating the nucleic acid molecule with a restriction enzyme; attaching one or more chemical groups or compounds to the nucleic acid; conjugating one or more chemical groups or compounds to the nucleic acid; incubating nucleic acid molecules with an enzyme that modifies the nucleic acid molecules by adding or removing one or more elements, chemical groups, or compounds.

It is contemplated that a nucleic acid is isolated or extracted by any technique known to those of skill in the art, including, but not limited to, using a gel, column, matrix or filter to isolate the nucleic acids. In some embodiments, the gel is a polyacrylamide or agarose gel.

It is specifically contemplated that the sequence information of SMCHD1 gene could be obtained by any method known in the art, including, but not limited to, genome sequencing, exome sequencing, chain terminating sequencing, restriction digestion, allele-specific polymerase reaction, single-stranded conformational polymorphism analysis, genetic bit analysis, temperature gradient gel electrophoresis, or ligase chain reaction.

In additional embodiments, the methods described herein may further comprise identifying the subject as being a carrier of an FSHD2 mutation or at risk for FSHD2. In still further embodiments, methods described herein comprise providing genetic counseling regarding the risks of transmitting FSHD2 after identifying the subject as being a carrier of an FSHD2 mutation or at risk for FHSD2.

Certain embodiments provide methods for detecting facioscapulohumeral dystrophy (FSHDs) comprising assaying for SMCHD1 expression in a sample from the subject, determining that the sample has reduced SMCHD1 expression as compared to a SMCHD1 control or reference, and identifying the subject as having a biomarker for FSHDs. In some aspects, FSHDs may comprise FSHD1 and FSHD2. In certain embodiments, methods specifically involve identifying the subject as having a biomarker for FSHD2.

Certain embodiments provide methods for assaying for SMCHD1 expression by measuring SMCHD1 mRNA in a sample. mRNA expression can be measured and quantified by any method known in the art, including, but not limited to polymerase chain reaction (PCR). In certain aspects, mRNA expression is quantified by real time PCR.

Additional embodiments provide methods for assaying for SMCHD1 expression by measuring SMCHD1 protein in a sample. SMCHD1 protein expression can be measured or quantified by any method known in the art. Exemplary examples include, but not limited to immunologic detection or mass spectrometry.

In additional embodiments, methods are provided to detect FSHD in a subject by detecting reduced binding between SMCHD1 protein and D4Z4 array, as compared to a normal control. To determine and quantify the binding between SMCHD1 and D4Z4 array, all methods known in the art can be employed, including, but not limited to chromatin immunoprecipitation.

In still further embodiments, methods disclosed herein further comprise predicting, assessing and/or evaluating the severity of FSHD in a subject by detecting and/or measuring the presence of a SMCHD1 variant, SMCHD1 mRNA expression, SMCHD1 protein expression, or binding between SMCHD1 protein and D4Z4 array, respectively or a combination thereof, and comparing to normal controls.

In further embodiments, methods described herein further comprise diagnosing FSHD in a subject by detecting the presence of a SMCHD1 mutation or a SMCHD1 variant as described herein, reduced SMCHD1 mRNA expression, detecting reduced SMCHD1 protein expression, or detecting reduced binding between SMCHD1 protein and D4Z4 array, as compared to normal controls, respectively, or a combination thereof. As used herein, normal control means a subject or a sample from a subject which carries a normal/wild type SMCHD1 gene.

In additional embodiments, methods described herein comprise diagnosing FSHD in a subject by detecting the presence of a SMCHD1 mutation or a SMCHD1 variant as described herein, reduced SMCHD1 mRNA expression, detecting reduced SMCHD1 protein expression, or detecting reduced binding between SMCHD1 protein and D4Z4 array, as compared to normal controls, respectively, or a combination thereof, in a sample from a prenatal embryo, or in an in vitro fertilization egg prior to pre-implantation.

In still further embodiments, methods described herein comprise providing genetic counseling regarding the risks of transmitting FSHD to a subject after detecting the presence of a SMCHD1 variant as described herein, detecting reduced SMCHD1 mRNA expression, detecting reduced SMCHD1 protein expression, or detecting reduced binding between SMCHD1 protein and D4Z4 array, as compared to normal controls, respectively, or a combination thereof in the subject.

In certain embodiments, methods for detecting a variant of SMCHD1 gene associates with FSHD2 in a subject. In additional embodiments, methods for detecting a variant of SMCHD1 gene in a subject which is at risk of developing FSHD or exhibiting symptoms of FSHD are provided. It is contemplated that the presence of said variant of SMCHD1 gene reduces SMCHD1 protein levels in a subject as compared to a normal control. The variant of SMCHD1 gene associates with FSHDs includes, but is not limited to, the variants as described herein.

Therapeutic methods for treating FSHD, including both FSHD1 and FSHD2, are contemplated. Certain embodiments are directed to methods for treating FSHD by administering to a subject in need thereof a pharmaceutical composition comprising a compound that provides SMCHD1 activity to cells of the subject. In some embodiments, the compound comprises a polypeptide of at least 70%, 75%, 80%, 85%, 90%, 95%, 96%, 97%, 98%, or 99% or higher sequence identity, including all values and ranges there between, or any range derivable therein, compared to a SMCHD1 protein (Genebank accession number: NP_{—}056110).

In another embodiments, the compound comprises a vector comprising a promoter operably linked to a nucleic acid segment encoding a polypeptide that is 70%, 75%, 80%, 85%, 90%, 95%, 96%, 97%, 98%, or 99% or higher sequence identity, including all values and ranges there between, or any range derivable therein, compared to a SMCHD1 protein (Genebank accession number: NP_{—}056110). In further embodiments, the nucleic acid segment is at least 70%, 75%, 80%, 85%, 90%, 95%, 96%, 97%, 98%, or 99% or higher sequence identity, including all values and ranges there between, or any range derivable therein, compared to the nucleic acid sequence of Genebank accession number: NM_{—}015295.2.

Certain embodiments involve methods for diagnosing a subject, comprising obtaining a sample from a subject, receiving information of the level of SMCHD1 expression in the sample as compared to a control or reference level, and diagnosing the subject as having or being at risk for FSHD2 after determining that the level of SMCHD1 expression is reduced compared to the control or reference level. Methods for treating a subject with FSHD2 after being diagnosed with FSHD2 is also contemplated.

Samples are biological samples containing cells, proteins, and/or nucleic acids. In some embodiments, the sample is obtained from an adult subject (in humans, someone 18 years old or older), while in other embodiments, the sample is obtained from a subject that is not yet an adult. In certain embodiments, the subject is a fetus or embryo. In particular embodiments, the biological sample comprises DNA extracted from fetal or amniotic or maternal cells. In some embodiments, the cells are obtained by amniocentesis or chronic villus sampling. In some cases, the cells are obtained after about 10 to about 12 weeks of gestation or after about 15 to about 18 weeks after gestation. A biological sample may be from blood, plasma, serum, urine, saliva, tears, tissue or other biopsy.

In further embodiments, methods may also involve determining the presence or absence of a polymorphism resulting in a functional polyadenylation sequence operationally linked to exon 3 of the DUX4 gene. The determination may involve genotyping a biological sample. A determination of the absence of a functional polyadenylation sequence operationally linked to exon 3 may indicate the subject does not have a genetic predisposition to develop or is not suffering from FSHD, while the presence of the sequence may indicate a predisposition toward developing the disease (or the presence of the disease already). In certain embodiments, the polymorphism is described in PCT/US2011/048318, which has been published as WO 2012/024535, which is hereby incorporated by reference.

Embodiments also concern kits, which may be in a suitable container, that can be used to achieve the described methods. In some embodiments, there are kits for detecting FSHD in a subject comprising an agent for detecting the presence of a variant of SMCHD1 as described herein. Kits for detecting FSHD in a subject, comprising an agent for detecting reduced SMCHD1 mRNA expression, an agent for detecting reduced SMCHD1 protein expression, an agent for detecting reduced SMCHD1 protein activity, or an agent for detecting reduced binding between SMCHD1 and D4Z4 arrays in a sample from the subject are also contemplated. In further embodiments, a kit may include one or more buffers, such as buffers for nucleic acids or for reactions involving nucleic acids. In still further embodiments, a kit may include one or more enzymes, such as a polymerase or a restriction enzyme. Kits may also include nucleotides for use with the polymerase.

In methods and compositions described herein, the subject is a mammal. In some aspects, the subject is a human. In some embodiments, the sample is a biological sample, including, but not limited to a blood sample, a urine sample, a body fluid sample, or a tissue sample. In certain embodiments, the sample is a blood sample. In certain embodiments, the biological sample is from a patient. In further embodiments, the patient is a human patient. In additional aspects, the subject maybe a pre-natal embryo or fetus or an in vitro fertilized egg prior to implantation.

Additional embodiments include reporting to the subject or patient or to a treating clinician the results of any analysis or determination. Such reporting can involve an electronic or physical document.

Further embodiments may involve knowing that a patient or subject is at risk for FSHD based on an analysis or determination discussed herein and subsequently treating or counseling the patient accordingly. A clinician may discuss lifestyle options to minimize muscle damage, career counseling, and/or genetic counseling. These things may occur after a subject or patient is identified as having or being at risk for FSHD.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1a-1c. Schematic of the FSHD locus. Different combinations of D4Z4 chromatin relaxation are shown with the associated chromosomal context and patient phenotype. The D4Z4 array is shown as a series of white triangles on chromosome 4. The homologous array on chromosome 10 is depicted in grey. The FSHD permissive 4qA, and FSHD non-permissive 10q and 4qB haplotypes are depicted as white and light grey boxes, respectively. a, In the normal condition, D4Z4 arrays of >10 units are densely CpG methylated (black dots) on all four chromosomes. b, FSHD1 is associated with D4Z4 array contraction-dependent D4Z4 hypomethylation and DUX4 expression from the deleted chromosome having a FSHD-permissive 4qA haplotype. Permissive 4qA haplotypes have a DUX4 polyadenylation signal (pA) distal to the last unit of the D4Z4 array. This pA signal results in stabilization of DUX4 mRNA. Contraction-dependent chromatin relaxation on non-permissive haplotypes (4qB or 10q) do not cause disease, because they lack this DUX4 pA signal. In FSHD1, D4Z4 hypomethylation is restricted to the contracted array. FSHD2 is caused by D4Z4 array contraction-independent chromatin relaxation of a D4Z4 locus with a permissive haplotype. In this case all four D4Z4 arrays are hypomethylated, and the hypomethylation phenotype can segregate independently of the permissive 4q haplotype within a family. Thus, family members who inherit the hypomethylation phenotype without a permissive haplotype do not develop FSHD2 (CONTROL). Chromosome 10 arrays are not depicted. c, Representative immunofluorescence image of variegated production of the DUX4 protein in a subset of FSHD1¹³ (data not shown) and FSHD2 myonuclei. This ectopic DUX4 expression is thought to result in muscle damage, weakness, and other features of FSHD. DUX4 immunoreactivity (red) is seen in some nuclei (blue) in FSHD2 myotubes (green).

FIGS. 2a-2d: FSHD2 families with SMCHD1 variants. a, Pedigrees of FSHD2 families showing the independent segregation of D4Z4 hypomethylation and FSHD-permissive alleles. Only in those individuals in whom a permissive (P) allele combines with D4Z4 hypomethylation (<25%) can FSHD be diagnosed, while D4Z4 hypomethylated individuals carrying non-permissive (NP) alleles are unaffected. b, Pedigrees of sporadic (left panel) and familial (right panel) FSHD2 kindreds. Methylation analysis of the FseI site in D4Z4 shows the degree of methylation (left panels). SMCHD1 mRNA analysis in carriers of SMCHD1 variants and controls (C1-2) shows exon skipping or cryptic splice site usage (right panels). c, Western blot analysis of fibroblast cultures of 6 controls (C) and 8 individuals carrying a SMCHD1 variant (S). S# denotes member of family Rf854 with only a synonymous coding SNP. d, Bar diagram of ChIP analysis showing binding of SMCHD1 to D4Z4 but not to GAPDH (left panel) and reduced levels of SMCHD1 binding to D4Z4 (right panel) in FSHD2 patient 2305 from family Rf683 (FIG. 5). Error bars represent +/−1 standard deviation of duplicate experiments.

FIGS. 3a-3f: SMCHD1 haploinsufficiency results in DUX4 expression in normal human myoblasts. (a,b) shRNAs against different regions of SMCHD1 are effective in reducing the production of SMCHD1 in normal human primary myoblasts at the RNA and protein levels. Numbers below the graph and the gel lanes indicate the regions within the SMCHD1 transcript that are homologous to the indicated shRNAs. a, SMCHD1 mRNA levels were measured by quantitative RT-PCR and normalized to RPP30 transcript levels in a multiplexed reaction. Normalized SMCHD1 levels are shown as a percentage of the levels found in the same cells treated with a vector expressing a scrambled sequence. Error bars, s.d. of the mean for three separate reactions. b, Protein blot of protein samples from the cultures in a normalized to tubulin. c, Semiquantitative RT-PCR analysis of DUX4 in cells deficient in SMCHD1. GAPDH was amplified to confirm RNA integrity. d, Examples of DUX4 immunoreactive nuclei observed in myotubes where SMCHD1 levels were reduced using shRNA 4103 or 6051. Myotubes are shown with nuclei labeled with DAPI (blue) and stained for DUX4 (red). GFP fluorescence produced from the lentivirus vector expressing the shRNAs is also shown. Scale bars, 50 μm (e,f) Antisense oligonucleotide-mediated exon skipping of SMCHD1 exons 36 and 29 in normal human myoblasts 2333 and 2435. Cells were treated with antisense oligonucleotides designed to reproduce this skipping, and primers homologous to flanking exons (shown above each gel) were used to evaluate the proportion of transcripts with skipped exons. DUX4 expression from the same cells is shown below each panel of SMCHD1 exon analysis. Results are also shown for myotube RNA from affected individuals in both families with the mutations. e, An 184-bp fragment is produced when exon 36 is skipped. f, An 124-bp fragment is produced when exon 29 is skipped. *, low DUX4 expression levels consistent with inefficient SMCHD1 exon skipping. An antisense oligonucleotide targeting exon 50 of the DMD gene (encoding dystrophin) was used as a negative control.

FIGS. 4a-4e. Design and results of the D4Z4 methylation test. a, Overview of methylation analysis method. b, Example of methylation analysis in an FSHD2 family. Methylated (M) and unmethylated (UM) D4Z4 fragments are indicated. Below each lane the methylation value is indicated in %. Y indicates cross hybridizing Y fragment. The hypomethylated mother in this family is not affected in the absence of a permissive haplotype. c, Schematic of methylation test showing the p13E-11 probe region at the proximal end of the D4Z4 repeat array and the expected D4Z4 fragment sizes upon digestion with restriction enzymes EcoRI, BglII and FseI (EcoRI sites are not shown as they are outside the indicated area and the enzyme is only used for additional fragmentation of the gDNA). The position of the chromosome 10q-specific restriction enzyme BlnI (black bottom half) that was previously used for the chromosomes 4q only methylation analysis is indicated. d, Schematic of FseI methylation analysis for both chromosomes 4 (old method; left panel)4 and chromosomes 4 and 10 (new method; right panel). Bar diagram of average methylation levels in controls (N=17), FSHD1 patients (N=22) and FSHD2 patients (N=33) obtained by the old method (left panel) and same samples by new method (right panel). Error bar represents standard deviation. FSHD2 patients are significantly hypomethylated by this test compared to controls and FSHD1 patients (*: p<0.005). Note that FSHD1 patients have methylation levels in between controls (normal methylation at all 4 alleles) and FSHD2 (hypomethylation at all 4 alleles) due to the presence of one hypomethylated allele. e, FseI methylation values of 72 control, 93 FSHD1 and 53 FSHD2 gDNA samples. Error bar represents standard deviation. FSHD2 patients are significantly hypomethylated by this test compared to controls and FSHD1 patients (*: p<0.005).

FIG. 5. Pedigrees of FSHD2 families. Individuals used in the whole exome sequencing are indicated in grey boxes beneath the individual. For each individual their ID, size of the smallest D4Z4 array in units (U) on a permissive allele, their FseI methylation level (%) and whether they are SMCHD1 variant carrier (SMC) or not (CTR), is indicated. SMC# indicates coding synonymous SNP identified in Rf854.

FIG. 6. Schematic of the human SMCHD1 locus. All exons are indicated with boxes. Information about the SMCHD1 protein domains and antibody epitopes is also given. SMCHD1 variants identified in this study are documented with their (predicted) consequences. The position of the 5′ and 3′ splice sites with respect to the coding frame is also indicated.

FIG. 7. RT-PCR analysis of SMCHD1 RNA in controls (C) and carriers of SMCHD1 splice site variants in families Rf696, Rf392 and Rf1014. RT-PCR products were sequence verified. Schematics of alternative splice events are shown on top and primers used to determine splicing are indicated with arrows. The splicing changes in family Rf696 can also be observed at lower frequency in the controls indicating that this variant shifts the balance (compare unspliced product with spliced products).

FIG. 8. SMCHD1 relative expression levels in myoblasts and myotubes.

FIGS. 9a-9b Genetic and epigenetic characterization of three FSHD families (A) Pedigrees of the families with complete genetic data. Top: pedigrees are shown of all three families, individuals in light grey were not available. Below each individual information is given for the methylation level at the FseI site in the first units of the D4Z4 arrays (%) and the presence (SMC) or absence (CTR) of a SMCHD1 mutation. In addition, information is given for the size of the D4Z4 repeat arrays on chromosomes 4 (in units) and the distal variation (A or B). Shaded boxes indicate FSHD-permissive genetic features. For example, individual I-1 of family Rf1110 carries has an FseI methylation level of 8% and is carrier of a SMCHD1 mutation. He carries one D4Z4 repeat array of 9 units on a 4A chromosome and one array of 63 units on a 4A chromosome. Middle: Methylation data of all three families with genomic DNA digested with restriction enzymes EcoRI, BglII and methylation sensitive FseI. Methylated (M) and unmethylated (UM) D4Z4 fragments are indicated. Bottom: D4Z4 repeat sizing data of samples in middle panel by digestion of genomic DNA with EcoRI (E) or with EcoRI and BlnI (B), followed by hybridisation with probe p13E-11. Chromosome 4-derived D4Z4 arrays are BlnI resistant while chromosome 10-derived arrays are not. The disease associated 9 units arrays (35 kb) are indicated with an asterisk. The cross-hybridizing DNA fragment on the Y chromosome is labelled with Y. Marker lanes are indicated on the right and left of the gel. Individual I-II of family Rf1110 was run on a separate gel as indicated with the vertical hairline. (B) Sequence traces of the SMCHD1 mutations in all three families and in control samples. Exonic sequences are indicated in black and intronic sequences in white. The position of the heterozygous mutations are highlighted in yellow.

FIGS. 10a-10b Depletion of SMCHD1 in FSHD1 myoblast augments DUX4 expression SMCHD1 depletion in FSHD1 primary myotubes leads to upregulation of DUX4 and DUX4 target transcripts RFLP2B, TRIM43 and ZSCAN4. FSHD1 myoblast cell line was transduced with lentiviral particles harboring scrambled shRNA constructs or SMCHD1 targeting constructs. After myotube formation cells were harvested for protein and RNA isolation. (A) Western blot analysis shows that SMCHD1 protein is depleted in myotubes transducted with shRNA constructs against SMCHD1 and not with scrambled shRNA. Tubulin is used as a loading control. (B) Relative expression levels of DUX4 and DUX4 target genes RFLP2B, TRIM43 and ZSCAN4 in SMCHD1 depleted FSHD1 myotubes. Y axis depicts relative changes in expression levels corrected against housekeeping gene beta-glucuronidase after SMCHD1 depletion normalized to expression levels of samples without SMCHD1 depletion.

FIGS. 11a-d Post-transcriptional regulation of SMCHD1 in skeletal muscle. A. Western showing a decrease of SMCHD1 protein and an increase in alpha-actin in human myoblasts differentiated 0, 24, 48, and 72 hrs. Alpha actin is used as differentiation control and alpha tubulin as loading control. B. Western showing a decrease of SMCHD1 protein between 48, 72, and 94 hrs after MyoD transduction of primary human fibroblasts using two independent SMCHD1 antibodies. C. RT-qPCR of Myogenin and SMCHD1 mRNA in human muscle cells in growth medium (GM) or differentiated (DM) for 94 hrs. D. MG132 proteosome inhibitor added for six hours to human muscle cells in growth medium (GM) or between 90-96 hrs in differentiation medium (DM).

FIG. 12 Binding of SMCHD1 to D4Z4 in D4Z4-12.5 and D4Z4-2.5 mice. SMCHD1 ChIP was performed on MEFs of D4Z4-12.5, D4Z4-2.5 mice and WT littermates as negative controls, Relative enrichment in triplo experiments with error bars representing SEM is shown.

FIG. 13 Schematic of the human SMCHD1 gene. All exons are indicated with boxes. Information about the SMCHD1 protein domains and Hinge antibody epitope is also given. SMCHD1 mutations identified in this study are documented with their (predicted) consequences. The position of the 5′ and 3′ splice sites with respect to the coding frame is also indicated. Mutations that result in a frameshift are indicated by an asterisk.

### DETAILED DESCRIPTION OF THE INVENTION

Certain embodiments are directed to methods and compositions related to a SMCHD1 (Structural maintenance of chromosome flexible hinge domain-containing 1) variant. In certain aspects, an isolated DNA molecule comprising a non-genomic sequence of human SMCHD1 variant is contemplated. In a further aspect, an isolated nucleic acid fragment comprises a SMCHD1 gene variant. The presence of a SMCHD1 variant in a cell or a subject has the capacity of reducing SMCHD1 activity, such as reducing SMCHD1 mRNA expression, reducing SMCHD1 protein expression, reducing SMCHD1 protein activity, or reducing the binding between SMCHD1 and the D4Z4 array as compared to a cell or a subject with a wild-type SMCHD1 sequence. SMCHD1 variants in humans modify epigenetic repression and causes facioscapulohumeral dystrophy (FSHD), especially FSHD2. Methods for detecting and/or diagnosing FSHD by assaying the presence of SMCHD1 variant, the reduced activity of SMCHD1 are contemplated. In additional aspects, methods for treating a patient with FSHD by administering a compound that provides SMCHD1 activity to the patient are provided. Specifically, the compound may be a polypeptide of SMCHD1 or a nucleic acid segment encoding SMCHD1. Kits used to achieve the methods described herein are also contemplated.

### I. DEFINITIONS

As used herein, the term “gene,” “nucleic acid”, “polynucleotide,” “sequence,” “fragment, or “segment,” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide may be chimeric, i.e., composed of heterologous molecules.

A “vector” or “construct” (sometimes referred to as gene delivery system or gene transfer “vehicle”) refers to a macromolecule or complex of molecules comprising a polynucleotide to be delivered to a host cell, either in vitro or in vivo.

A “plasmid”, a common type of a vector, is an extra-chromosomal DNA molecule separate from the chromosomal DNA which is capable of replicating independently of the chromosomal DNA. In certain cases, it is circular and double-stranded.

A “viral vector” refers to a virus capable of delivering a polynucleotide into a host cell, either in vivo or in vitro. Commonly used viral vectors include, but are not limited to, retroviruses, lentiviruses, and adenoviruses.

The term “cell” is herein used in its broadest sense in the art and refers to a living body which is a structural unit of tissue of a multicellular organism, is surrounded by a membrane structure which isolates it from the outside, has the capability of self replicating, and has genetic information and a mechanism for expressing it. Cells used herein may be naturally-occurring cells or artificially modified cells (e.g., fusion cells, genetically modified cells, etc.).

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” refers in a moiety capable of detection, such as a radioactive isotope or group containing same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “hybrid” refers to the product of a hybridization procedure between a probe and a target. The term “hybridizing conditions” has general reference to the combinations of conditions that are employable in a given hybridization procedure to produce hybrids, such conditions typically involving controlled temperature, liquid phase, and contact between a probe (or probe composition) and a target. Conveniently and preferably, at least one denaturation step precedes a step wherein a probe or probe composition is contacted with a target. Guidance for performing hybridization reactions can be found in Ausubel et al. (2003). Aqueous and nonaqueous methods are described in that reference and either can be used. Hybridization conditions may be a 50% formamide, 2×SSC wash for 10 minutes at 45° C. followed by a 2×SSC wash for 10 minutes at 37° C.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 30%, e.g., at least 40%, 50%, 60%, 70%, 80%, 90% or 100%, of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a Blossum 62 scoring matrix with a gap penalty of 12, a gap extend penalty of 4, and a frameshift gap penalty of 5.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### II. FSHD

There are two forms of facioscapulohumeral muscular dystrophy, FSHD1 and FSHD2, that converge at the level of somatic relaxation of the D4Z4 chromatin structure and transcriptional derepression of the DUX4 retrogene encoded by the D4Z4 repeat unit. FSHD is the second most common myopathy in adults affecting the lives and daily activities of >15,000 people in the US. FSHD patients suffer from progressive and irreversible weakness and wasting of the facial and upper extremity muscles. With disease progression other skeletal muscles may also become affected. The penetrance is highly variable: ˜20% of carriers remain asymptomatic while an equal proportion becomes wheelchair-dependent. The disease onset is usually in the second decade, but ˜5% patients are diagnosed before the age of 10. In >50% of patients, there is an asymmetric distribution of muscle involvement. Patients also may suffer from sensorineural hearing loss and retinovasculopathy, pain and fatigue. In severely affected patients, mental retardation and epilepsy has been reported (Tawil & van der Maarel, 2006). There is no cure for FSHD.

In most patients FSHD is caused by a contraction of the polymorphic D4Z4 repeat array on chromosome 4q to an array of 1-10 units (FSHD1), but only if this contraction occurs on a specific genetic background of the 4q subtelomere (Lemmers, et al., 2004; Lemmers, et al., 2007; Lemmers, et al., 2010a; Lemmers, et al., 2010b; Wijmenga, et al., 1992). Contracted D4Z4 arrays have a reduced repressive chromatin structure facilitating the transcriptional derepression of the non-polyadenylated DUX4 retrogene encoded by each D4Z4 unit (Lemmers, et al., 2010a; Dixit, et al., 2007; Snider, et al., 2010; van Overveld, et al., 2003; Zeng, et al., 2009; Cabianca, et al., 2012;). Only from the telomeric unit of the array, the two exons of DUX4 can be spliced to a third exon immediately distal to the array that provides the DUX4 transcript with a stabilizing polyadenylation (pA) signal (Lemmers, et al., 2010a; Dixit, et al., 2007; Snider, et al., 2010). Chromosomal backgrounds on which repeat contractions do not cause FSHD lack this exon or pA signal rendering support to the model that stabilization of the DUX4 transcript is pivotal in FSHD pathogenesis (Lemmers, et al., 2010a). Positional cloning studies in FSHD patients with unusual D4Z4 repeat structures further support this disease model (Lemmers, et al., 2010a). Indeed, while a variegated pattern of DUX4 protein expression can be observed in FSHD myotubes, DUX4 expression in normal myotubes is absent or strongly reduced (Snider, et al., 2010). Thus, FSHD is caused by inefficient repression of the DUX4 retrogene leading to inappropriate bursts of DUX4 protein expression in skeletal muscle (van der Maarel, et al., 2011).

### III. SMCHD1

SMCHD1 protein is a non-canonical member of the SMC superfamily that includes core cohesion and condensin subunits. SMCHD1 is a novel factor that was identified in a screen for dominant mutations that modify expression of an autosomal transgene in mouse. When bred to homozygosity SMCHD1 mutation results in female specific embryo lethality, indicating a role in X inactivation (Blewitt et al., 2005). It has been shown that SMCHD1 localizes to the inactive X chromosome, but in SMCHD1 mutants, DNA methylation of CpG islands, a key modification required for maintenance of X inactivation, is absent. These results suggest SMCHD1 protein's role in maintaining X inactivation (Blewitt et al., 2008).

The SmcHD1 mutation was originally called the Momme D1 (Modifiers of Murine Metastable Epialleles D1) locus (Blewitt et al., 2005). The term metastable epiallele has been applied to genes that show variable expression because of probabilistic determinants of epigenetic repression (Rakyan et al., 2002). An example of a metastable epiallele in mice is the agouti viable yellow (A^{vy}) locus; coat colors of isogenic mice can vary based on the epigenetic state of a retrotransposon integrated near the agouti promoter (Duhl et al., 1994). SmcHD1 is a modifier of metastable epialleles because SmcHD1 haploinsufficiency increased the penetrance of agouti expression (Blewitt et al., 2005). As discussed herein, the inventors determined that SMCHD1 can act as a modifier in FSHD1.

In certain aspects, the SMCHD1 protein is a human SMCHD1 protein having the Genebank accession number NP_{—}056110.

### IV. NUCLEIC ACID

Certain embodiments provide an isolated DNA molecule comprising a non-genomic sequence of human SMCHD1 which comprises a SMCHD1 gene variant. Additional embodiments involve an isolated nucleic acid fragment comprising a SMCHD1 gene variant. The SMCHD1 gene variant includes any SMCHD1 gene variant, such as deletion variants, missense variant, splice-site variant, that reduces SMCHD1 activity in a cell as compared to a wild-type SMCHD1 sequence. The SMCHD1 gene variant comprises any variant as disclosed herein. The SMCHD1 gene variant may comprises one or more mutations as disclosed herein.

In additional embodiments, the isolated DNA molecule or nucleic acid fragment is modified by a label. The label can be any label that is detected, or is capable of being detected. Examples of suitable labels include, e.g., chromogenic label, a radiolabel, a fluorescent label, and a biotinylated label. The term “chromogenic label” includes all agents that have a distinct color or otherwise detectable marker. In addition to chemical structures having intrinsic, readily-observable colors in the visible range, other markers used include fluorescent groups, biotin tags, enzymes (that may be used in a reaction that results in the formation of a colored product), magnetic and isotopic markers, and so on. The foregoing list of detectable markers is for illustrative purposes only, and is in no way intended to be limiting or exhaustive.

The label may be attached to the agent using methods known in the art. Labels include any detectable group attached to the glucose molecule, or detection agent that does not interfere with its function. Further labels that may be used include fluorescent labels, such as Fluorescein, Texas Red, Lucifer Yellow, Rhodamine, Nile-red, tetramethyl-rhodamine-5-isothiocyanate, 1,6-diphenyl-1,3,5-hexatriene, cis-Parinaric acid, Phycoerythrin, Allophycocyanin, 4′,6-diamidino-2-phenylindole (DAPI), Hoechst 33258, 2-aminobenzamide, and the like. Further labels include electron dense metals, such as gold, ligands, haptens, such as biotin, radioactive labels.

A fluorophore contains or is a functional group that will absorb energy of a specific wavelength and re-emit energy at a different (but equally specific) wavelength. The amount and wavelength of the emitted energy depend on both the fluorophore and the chemical environment of the fluorophore. Fluorophores can be attached to protein using functional groups and or linkers, such as amino groups (Active ester, Carboxylate, Isothiocyanate, hydrazine); carboxyl groups (carbodiimide); thiol (maleimide, acetyl bromide); azide (via click chemistry or non-specifically (glutaraldehyde).

Fluorophores can be proteins, quantum dots (fluorescent semiconductor nanoparticles), or small molecules. Common dye families include, but are not limited to Xanthene derivatives: fluorescein, rhodamine, Oregon green, eosin, Texas red etc.; Cyanine derivatives: cyanine, indocarbocyanine, oxacarbocyanine, thiacarbocyanine and merocyanine; Naphthalene derivatives (dansyl and prodan derivatives); Coumarin derivatives; oxadiazole derivatives: pyridyloxazole, nitrobenzoxadiazole and benzoxadiazole; Pyrene derivatives: cascade blue etc.; BODIPY (Invitrogen); Oxazine derivatives: Nile red, Nile blue, cresyl violet, oxazine 170 etc.; Acridine derivatives: proflavin, acridine orange, acridine yellow etc.; Arylmethine derivatives: auramine, crystal violet, malachite green; CF dye (Biotium); Alexa Fluor (Invitrogen); Atto and Tracy (Sigma Aldrich); FluoProbes (Interchim); Tetrapyrrole derivatives: porphin, phtalocyanine, bilirubin; cascade yellow; azure B; acridine orange; DAPI; Hoechst 33258; lucifer yellow; piroxicam; quinine and anthraqinone; squarylium; oligophenylenes; and the like.

Other fluorophores include: Hydroxycoumarin; Aminocoumarin; Methoxycoumarin; Cascade Blue; Pacific Blue; Pacific Orange; Lucifer yellow; NBD; R-Phycoerythrin (PE); PE-Cy5 conjugates; PE-Cy7 conjugates; Red 613; PerCP; TruRed; Fluor X; Fluorescein; BODIPY-FL; TRITC; X-Rhodamine; Lissamine Rhodamine B; Texas Red; Allophycocyanin; APC-Cy7 conjugates.

Alexa Fluor dyes (Molecular Probes) include: Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, and Alexa Fluor 790.

Cy Dyes (GE Healthcare) include Cy2, Cy3, Cy3B, Cy3.5, Cy5, Cy5.5 and Cy7.

Nucleic acid probes include Hoechst 33342, DAPI, Hoechst 33258, SYTOX Blue, Chromomycin A3, Mithramycin, YOYO-1, Ethidium Bromide, Acridine Orange, SYTOX Green, TOTO-1, TO-PRO-1, TO-PRO: Cyanine Monomer, Thiazole Orange, Propidium Iodide (PI), LDS 751, 7-AAD, SYTOX Orange, TOTO-3, TO-PRO-3, and DRAQ5.

Cell function probes include Indo-1, Fluo-3, DCFH, DHR, SNARF.

Fluorescent proteins include Y66H, Y66F, EBFP, EBFP2, Azurite, GFPuv, T-Sapphire, Cerulean, mCFP, ECFP, CyPet, Y66W, mKeima-Red, TagCFP, AmCyan1, mTFP1, S65A, Midoriishi Cyan, Wild Type GFP, S65C, TurboGFP, TagGFP, S65L, Emerald, S65T (Invitrogen), EGFP (Clontech), Azami Green (MBL), ZsGreen1 (Clontech), TagYFP (Evrogen), EYFP (Clontech), Topaz, Venus, mCitrine, YPet, TurboYFP, ZsYellowl (Clontech), Kusabira Orange (MBL), mOrange, mKO, TurboRFP (Evrogen), tdTomato, TagRFP (Evrogen), DsRed (Clontech), DsRed2 (Clontech), mStrawberry, TurboFP602 (Evrogen), AsRed2 (Clontech), mRFP1, J-Red, mCherry, HcRedl (Clontech), Katusha, Kate (Evrogen), TurboFP635 (Evrogen), mPlum, and mRaspberry.

### V. DETECTING SMCHD1

In some embodiments, methods for detecting a mutation associated with FSHD are provided, comprising assaying for the presence of a variant in one or both alleles of a SMCHD1 gene. The variant can be any variant which bears a mutation or polymorphism associated with a reduction of SMCHD1 activity as compared to a wild type SMCHD1 gene, including, but not limited to the mutations or variants disclosed herein.

Determining a variant of SMCHD1 gene can, but need not, include obtaining a sample comprising nucleic acid from a subject, and/or assessing the identity, presence or absence of one or more mutations.

Samples that are suitable for use in the methods described herein contain genetic material, e.g., genomic DNA (gDNA). Non-limiting examples of sources of samples include urine, blood, cells, and tissues. The sample itself will typically consist of nucleated cells (e.g., blood or buccal cells), tissue, etc., removed from the subject. The subject can be an adult, a child, a fetus, or an embryo. In some embodiments, the sample is obtained prenatally, either from a fetus or an embryo or from the mother (e.g., from fetal or embryonic cells in the maternal circulation). Methods and reagents are known in the art for obtaining, processing, and/or analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

The sample may be further processed before the detecting step. For example, DNA or RNA in a cell or tissue sample can be separated from other components of the sample. The sample can be concentrated and/or purified to isolate DNA or RNA. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA or RNA. See, e.g., Ausubel et al., 2003, supra. All samples obtained from a subject, including those subjected to any sort of further processing, are considered to be obtained from the subject.

The presence or absence of a SMCHD1 gene variant may be determined by any methods known in the art, e.g., gel electrophoresis, capillary electrophoresis, size exclusion chromatography, sequencing, and/or arrays to detect the presence or absence of the marker(s) of the haplotype. Amplification of nucleic acids, where desirable, can be accomplished using methods known in the art, e.g., PCR.

Methods of nucleic acid analysis to detect mutation, polymorphisms and/or polymorphic variants include, e.g., microarray analysis. Hybridization methods, such as Southern analysis, or fluorescent intensity analysis of microarrays, can also be used (see Ausubel et al., 2003; Redon et al., 2006).

Other methods include direct manual sequencing (Church and Gilbert, 1984; Sanger et al., 1977; U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR. See, e.g., U.S. Patent Publication No. 2004/0014095, to Gerber et al., which is incorporated herein by reference in its entirety. In some embodiments, the sequence is determined on both strands of DNA.

In order to detect mutation and/or polymorphisms and/or polymorphic variants, it will frequently be desirable to amplify a portion of genomic DNA encompassing the mutation/polymorphic site. Such regions can be amplified and isolated by PCR using oligonucleotide primers designed based on genomic and/or cDNA sequences that flank the site. See e.g., PCR Primer: A Laboratory Manual; McPherson et al., 2000; Mattila et al., 1991; Eckert et al., 1991; and U.S. Pat. No. 4,683,202. Other amplification methods that may be employed include the ligase chain reaction (LCR) (Wu and Wallace, 1989, Landegren et al., 1988), transcription amplification (Kwoh et al., 1989), self-sustained sequence replication (Guatelli et al., 1990), and nucleic acid based sequence amplification (NASBA). Guidelines for selecting primers for PCR amplification are well known in the art. See, e.g., McPherson et al. (2000). A variety of computer programs for designing primers are available, e.g., “Oligo” (National Biosciences, Inc, Plymouth Minn.), MacVector (Kodak/IBI), and the GCG suite of sequence analysis programs (Genetics Computer Group, Madison, Wis. 53711).

In one example, a sample (e.g., a sample comprising genomic DNA), is obtained from a subject. The DNA in the sample is then examined to detect the presence of a SMCHD1 variant as described herein. The detection of a SMCHD1 variant can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular variant.

In some embodiments, restriction digest analysis can be used to detect the existence of a mutation or a polymorphic variant of SMCHD1 gene, if the mutation or alternate polymorphic variants result in the creation or elimination of a restriction site. A sample containing genomic DNA is obtained from the individual. Polymerase chain reaction (PCR) can be used to amplify a region comprising the mutation site or polymorphic site, and restriction fragment length polymorphism analysis is conducted (see Ausubel et al., supra). The digestion pattern of the relevant DNA fragment may indicate the presence or absence of a particular polymorphic variant or a particular mutation variant of SMCHD1 and may be indicative of FSHD.

Sequence analysis can also be used to detect specific variants. A sample comprising DNA or RNA is obtained from the subject. PCR or other appropriate methods can be used to amplify a portion encompassing the variant site, such as mutation or polymorphism sites, if desired. The sequence is then ascertained, using any standard method, and the presence of a polymorphic variant is determined.

DNA containing the amplified portion may be dot-blotted, using standard methods (see Ausubel et al., supra), and the blot contacted with the oligonucleotide probe. The presence of specific hybridization of the probe to the DNA is then detected.

Allele-specific oligonucleotides can be used to detect the presence of a polymorphic or a mutation variant, e.g., through the use of dot-blot hybridization of amplified oligonucleotides with allele-specific oligonucleotide (ASO) probes (see, for example, Saiki et al., 1986). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism or a mutation. An allele-specific oligonucleotide probe can be prepared using standard methods (see Ausubel et al., supra).

Real-time pyrophosphate DNA sequencing is yet another approach to detection of mutation variants, or polymorphisms and polymorphic variants (Alderborn et al., 2000). Additional methods include, for example, PCR amplification in combination with denaturing high performance liquid chromatography (dHPLC) (Underhill et al., 1997).

The methods can include determining the genotype of a subject with respect to both copies of the mutation or polymorphic site present in the genome. For example, the complete genotype may be characterized as −/−, as −/+, or as +/+, where a plus sign indicates the presence of the variant of interest, and a minus sign indicates the absence of the variant of interest and/or the presence of the other or wild type sequence at the corresponding site. Any of the detection means described herein can be used to determine the genotype of a subject with respect to one or both copies of the polymorphism or mutations present in the subject's genome.

In some embodiments, it is desirable to employ methods that can detect the presence of multiple mutations or polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple mutation or polymorphic variants (e.g., polymorphic variants at a plurality of polymorphic sites) in parallel or substantially simultaneously according to certain embodiments of the invention.

Embodiments also contemplate methods for detecting FSHD in a subject by assaying for SMCHD1 expression in a sample. The SMCHD1 expression is assayed by measuring SMCHD1 mRNA, or SMCHD1 protein in the sample. The measurement and quantification of mRNA and protein can be any method or technique known in the art. For example, mRNA can be quantified by real time PCR; protein can be quantified by conventional immunologic detection method or mass spectrometry.

There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. The term “antibody” also refers to any antibody-like molecule that has an antigen binding region, and includes antibody fragments such as Fab′, Fab, F(ab′)2, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A Laboratory Manual, Cold Spring Harbor Laboratory, 1988; incorporated herein by reference).

In some embodiments, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot. The steps of various useful immunodetection methods have been described in the scientific literature, such as, e.g., Doolittle & Ben-Zeev, 1999; Gulbis & Galand, 1993; De Jager et al., 1993; and Nakamura et al., 1987, each incorporated herein by reference. Other methods for measuring protein expression are also contemplated, such as immunochromatography and 2D-gel electrophoresis. It will be readily appreciated that detection is not limited to the techniques described herein, and Western blotting, dot blotting, FACS analyses, and the like may also be used.

Some embodiments provide methods for detecting FSHD in a subject by assaying for binding between SMCHD1 and D4Z4 arrays. The binding can be assessed by any technique known in the art, such as immunoprecipitation. In particular embodiments, chromatin immunoprecipitation is contemplated.

In some aspects, there are provided methods for diagnosing FSHD when reduced mRNA expression of SMCHD1, or reduced protein expression of SMCHD1 or reduced binding between SMCHD1 protein and D4Z4 or a combination thereof are detected in a subject as compared to a control or a reference.

### VI. METHYLATION ANALYSIS

In additional aspects, the diagnosing methods for FSHD described herein may be combined with detecting other biomarkers of FSHD, including, but not limited to DUX4 expression, DNA hypomethylation of the D4Z4 array, or decreased repressive heterochromatin of the D4Z4 array.

The methylation state of a region of a genomic DNA, such as D4Z4 array, can be analyzed by using methylation sensitive endonuclease. In certain aspects, the methods are directed to analyzing the methylation state of CpGs island in a region of genomic DNA. A “CpG island” as used herein refers to regions of DNA with a high G/C content and a high frequency of CpG dinucleotides relative to the whole genome of an organism of interest. Also used interchangeably in the art is the term “CG island.” The ‘p’ in “CpG island” refers to the phosphodiester bond between the cytosine and guanine nucleotides.

Certain embodiments include digesting genomic DNA by several restriction enzymes followed by a conventional bisulfite treatment which is performed according to methods that are well known in the art. As a result, unmethylated cytosine residues are converted to Uracil residues, which in a subsequent sequencing reaction base calling are identified as “T” instead of “C”, when compared with a non bisulfite treated reference. Subsequent to bisulfite treatment, the sample is subjected to a conventional sequencing protocol.

The bisulfite treatment can be done according to standard methods that are well known in the art (Frommer et al., 1992; Zeschnigk et al., 1997; Clark et al., 1994). The sample can be purified, for example by a Sephadex size exclusion column or, at least by means of precipitation. It is also within the scope of some embodiments, if directly after bisulfite treatment, or directly after bisulfite treatment followed by purification, the sample is amplified by means of performing a conventional PCR using appropriate amplification primers. In some aspects, methylation dependent PCR is contemplated. For example, primers that only recognize bisulfate converted template, but not the non bisulfate treated template, may be used to distinguish methylation sites in a sample.

Proper restriction enzymes can be selected by a person skilled in the art. In one embodiment, FseI is used to analyze the D4Z4 methylation state. In additional embodiments, a combination of several restriction enzymes is selected in assaying methylation state of a region of genomic DNA. In one aspect, EcoRI, BulII and FseI are used.

The table below summarizes methylation sensitivity for NEB restriction enzymes, indicating whether or not cleavage is blocked or impaired by Dam, Dcm or CpG methylation if or when it overlaps each recognition site. REBASE, the restriction enzyme database, can be consulted for more detailed information and specific examples. (Marinus and Morris, 1973; Geier and Modrich, 1979; May and Hattman, 1975; Siegfried and Cedar, 1997).

| Enzyme | Sequence | Dam | Dcm | CpG |
|---|---|---|---|---|
| AatII | GACGT/C | Not Sensitive | Not Sensitive | Blocked |
| Acc65I | G/GTACC | Not Sensitive | Blocked by Some | Blocked by |
| | | | Overlapping | Some |
| | | | Combinations | Overlapping |
| | | | | Combinations |
| AccI | GT/MKAC | Not Sensitive | Not Sensitive | Blocked by |
| | | | | Overlapping |
| | | | | Methylation |
| AciI | CCGC(−3/−1) | Not Sensitive | Not Sensitive | Blocked |
| AclI | AA/CGTT | Not Sensitive | Not Sensitive | Blocked |
| AcuI | CTGAAG(16/14) | Not Sensitive | Not Sensitive | Not Sensitive |
| AfeI | AGC/GCT | Not Sensitive | Not Sensitive | Blocked |
| AflII | C/TTAAG | Not Sensitive | Not Sensitive | Not Sensitive |
| AflIII | A/CRYGT | Not Sensitive | Not Sensitive | Not Sensitive |
| AgeI | A/CCGGT | Not Sensitive | Not Sensitive | Blocked |
| AgeI-HF ™ | A/CCGGT | — | — | — |
| AhdI | GACNNN/NNGTC | Not Sensitive | Not Sensitive | Impaired by Some |
| | | | | Overlapping |
| | | | | Combinations |
| AleI | CACNN/NNGTG | Not Sensitive | Not Sensitive | Impaired by Some |
| | | | | Overlapping |
| | | | | Combinations |
| AluI | AG/CT | Not Sensitive | Not Sensitive | Not Sensitive |
| AlwI | GGATC(4/5) | Blocked | Not Sensitive | Not Sensitive |
| AlwNI | CAGNNN/CTG | Not Sensitive | Blocked by | Not Sensitive |
| | | | Overlapping | |
| | | | Methylation | |
| ApaI | GGGCC/C | Not Sensitive | Blocked by | Blocked by |
| | | | Overlapping | Overlapping |
| | | | Methylation | Methylation |
| ApaLI | G/TGCAC | Not Sensitive | Not Sensitive | Blocked by |
| | | | | Overlapping |
| | | | | Methylation |
| ApeKI | G/CWGC | Not Sensitive | Not Sensitive | Not Sensitive |
| ApoI | R/AATTY | Not Sensitive | Not Sensitive | Not Sensitive |
| AscI | GG/CGCGCC | Not Sensitive | Not Sensitive | Blocked |
| AseI | AT/TAAT | Not Sensitive | Not Sensitive | Not Sensitive |
| AsiSI | GCGAT/CGC | Not Sensitive | Not Sensitive | Blocked |
| AvaI | C/YCGRG | Not Sensitive | Not Sensitive | Blocked |
| AvaII | G/GWCC | Not Sensitive | Blocked by | Blocked by |
| | | | Overlapping | Overlapping |
| | | | Methylation | Methylation |
| AvrII | C/CTAGG | Not Sensitive | Not Sensitive | Not Sensitive |
| BaeGI | GKGCM/C | Not Sensitive | Not Sensitive | Not Sensitive |
| BaeI | (10/15)ACNNNNGTAYC(12/7) | Not Sensitive | Not Sensitive | Blocked by Some |
| | (SEQ ID NO: 2) | | | Overlapping |
| | | | | Combinations |
| BamHI | G/GATCC | Not Sensitive | Not Sensitive | Not Sensitive |
| BamHI- | G/GATCC | Not Sensitive | Not Sensitive | Not Sensitive |
| HF ™ | | | | |
| BanI | G/GYRCC | Not Sensitive | Blocked by Some | Blocked by Some |
| | | | Overlapping | Overlapping |
| | | | Combinations | Combinations |
| BanII | GRGCY/C | Not Sensitive | Not Sensitive | Not Sensitive |
| BbsI | GAAGAC(2/6) | Not Sensitive | Not Sensitive | Not Sensitive |
| BbvCl | CCTCAGC(−5/−2) | Not Sensitive | Not Sensitive | Impaired by |
| | | | | Overlapping |
| | | | | Methylation |
| BbvI | GCAGC(8/12) | Not Sensitive | Not Sensitive | Not Sensitive |
| BccI | CCATC(4/5) | Not Sensitive | Not Sensitive | Not Sensitive |
| BceAI | ACGGC(12/14) | Not Sensitive | Not Sensitive | Blocked |
| BcgI | (10/12)CGANNNNNNTGC(12/10) | Blocked by | Not Sensitive | Blocked by Some |
| | (SEQ ID NO: 3) | Overlapping | | Overlapping |
| | | Methylation | | Combinations |
| BciVI | GTATCC(6/5) | Not Sensitive | Not Sensitive | Not Sensitive |
| BclI | T/GATCA | Blocked | Not Sensitive | Not Sensitive |
| BfaI | C/TAG | Not Sensitive | Not Sensitive | Not Sensitive |
| BfuAI | ACCTGC(4/8) | Not Sensitive | Not Sensitive | Impaired by |
| | | | | Overlapping |
| | | | | Methylation |
| BfuCI | /GATC | Not Sensitive | Not Sensitive | Blocked by |
| | | | | Overlapping |
| | | | | Methylation |
| BglI | GCCNNNN/NGGC | Not Sensitive | Not Sensitive | Blocked by |
| | | | | Some |
| | | | | Overlapping |
| | | | | Combinations |
| BglII | A/GATCT | Not Sensitive | Not Sensitive | Not Sensitive |
| BlpI | GC/TNAGC | Not Sensitive | Not Sensitive | Not Sensitive |
| BmgBl | CACGTC(−3/−3) | Not Sensitive | Not Sensitive | Blocked |
| BmrI | ACTGGG(5/4) | Not Sensitive | Not Sensitive | Not Sensitive |
| BmtI | GCTAG/C | Not Sensitive | Not Sensitive | Not Sensitive |
| BpmI | CTGGAG(16/14) | Not Sensitive | Not Sensitive | Not Sensitive |
| Bpu10I | CCTNAGC(−5/−2) | Not Sensitive | Not Sensitive | Not Sensitive |
| BpuEI | CTTGAG(16/14) | Not Sensitive | Not Sensitive | Not Sensitive |
| BsaAI | YAC/GTR | Not Sensitive | Not Sensitive | Blocked |
| BsaBI | GATNN/NNATC | Blocked by | Not Sensitive | Blocked by Some |
| | | Overlapping | | Overlapping |
| | | Methylation | | Combinations |
| BsaHI | GR/CGYC | Not Sensitive | Blocked by Some | Blocked |
| | | | Overlapping | |
| | | | Combinations | |
| BsaI | GGTCTC(1/5) | Not Sensitive | Blocked by | Blocked by Some |
| | | | Overlapping | Overlapping |
| | | | Methylation | Combinations |
| BsaI- | GGTCTC(1/5) | — | Blocked by | — |
| HFT ™ | | | Overlapping | |
| | | | Methylation | |
| BsaJI | C/CNNGG | Not Sensitive | Not Sensitive | Not Sensitive |
| BsaWI | W/CCGGW | Not Sensitive | Not Sensitive | Not Sensitive |
| BsaXI | (9/12)ACNNNNNCTCC(10/7) | Not Sensitive | Not Sensitive | Not Sensitive |
| | (SEQ ID NO: 4) | | | |
| BseRI | GAGGAG(10/8) | Not Sensitive | Not Sensitive | Not Sensitive |
| BseYI | CCCAGC(−5/−1) | Not Sensitive | Not Sensitive | Blocked by |
| | | | | Overlapping |
| | | | | Methylation |
| BsgI | GTGCAG(16/14) | Not Sensitive | Not Sensitive | Not Sensitive |
| BsiEI | CGRY/CG | Not Sensitive | Not Sensitive | Blocked |
| BsiHKAI | GWGCW/C | Not Sensitive | Not Sensitive | Not Sensitive |
| BsiWI | C/GTACG | Not Sensitive | Not Sensitive | Blocked |
| BslI | CCNNNNN/NNGG | Not Sensitive | Blocked by Some | Blocked by Some |
| | | | Overlapping | Overlapping |
| | | | Combinations | Combinations |
| BsmAI | GTCTC(1/5) | Not Sensitive | Not Sensitive | Blocked by |
| | | | | Some |
| | | | | Overlapping |
| | | | | Combinations |
| BsmBI | CGTCTC(1/5) | Not Sensitive | Not Sensitive | Blocked |
| BsmFI | GGGAC(10/14) | Not Sensitive | Blocked by | Blocked by |
| | | | Overlapping | Overlapping |
| | | | Methylation | Methylation |
| BsmI | GAATGC(1/−1) | Not Sensitive | Not Sensitive | Not Sensitive |
| BsoBI | C/YCGRG | Not Sensitive | Not Sensitive | Not Sensitive |
| Bsp1286I | GDGCH/C | Not Sensitive | Not Sensitive | Not Sensitive |
| BspCNI | CTCAG(9/7) | Not Sensitive | Not Sensitive | Not Sensitive |
| BspDI | AT/CGAT | Blocked by | Not Sensitive | Blocked |
| | | Overlapping | | |
| | | Methylation | | |
| BspEI | T/CCGGA | Blocked by | Not Sensitive | Impaired |
| | | Overlapping | | |
| | | Methylation | | |
| BspHI | T/CATGA | Blocked by | Not Sensitive | Not Sensitive |
| | | Overlapping | | |
| | | Methylation | | |
| BspMl | ACCTGC(4/8) | Not Sensitive | Not Sensitive | Not Sensitive |
| BspQI | GCTCTTC(1/4) | Not Sensitive | Not Sensitive | Not Sensitive |
| BsrBI | CCGCTC(−3/−3) | Not Sensitive | Not Sensitive | Blocked by Some |
| | | | | Overlapping |
| | | | | Combinations |
| BsrDI | GCAATG(2/0) | Not Sensitive | Not Sensitive | Not Sensitive |
| BsrFI | R/CCGGY | Not Sensitive | Not Sensitive | Blocked |
| BsrGI | T/GTACA | Not Sensitive | Not Sensitive | Not Sensitive |
| BsrI | ACTGG(1/-1) | Not Sensitive | Not Sensitive | Not Sensitive |
| BssHII | G/CGCGC | Not Sensitive | Not Sensitive | Blocked |
| BssKI | /CCNGG | Not Sensitive | Blocked by | Blocked by |
| | | | Overlapping | Overlapping |
| | | | Methylation | Methylation |
| BssSI | CACGAG(−5/−1) | Not Sensitive | Not Sensitive | Not Sensitive |
| BstAPI | GCANNNN/NTGC | Not Sensitive | Not Sensitive | Blocked by Some |
| | | | | Overlapping |
| | | | | Combinations |
| BstBI | TT/CGAA | Not Sensitive | Not Sensitive | Blocked |
| BstEII | G/GTNACC | Not Sensitive | Not Sensitive | Not Sensitive |
| BstNI | CC/WGG | Not Sensitive | Not Sensitive | Not Sensitive |
| BstUI | CG/CG | Not Sensitive | Not Sensitive | Blocked |
| BstXI | CCANNNNN/NTGG | Not Sensitive | Blocked by Some | Not Sensitive |
| | | | Overlapping | |
| | | | Combinations | |
| BstYI | R/GATCY | Not Sensitive | Not Sensitive | Not Sensitive |
| BstZ17I | GTA/TAC | Not Sensitive | Not Sensitive | Blocked by Some |
| | | | | Overlapping |
| | | | | Combinations |
| Bsu36I | CC/TNAGG | Not Sensitive | Not Sensitive | Not Sensitive |
| BtgI | C/CRYGG | Not Sensitive | Not Sensitive | Not Sensitive |
| BtgZI | GCGATG(10/14) | Not Sensitive | Not Sensitive | Impaired |
| BtsCI | GGATG(2/0) | Not Sensitive | Not Sensitive | Not Sensitive |
| BtsI | GCAGTG(2/0) | Not Sensitive | Not Sensitive | Not Sensitive |
| Cac8I | GCN/NGC | Not Sensitive | Not Sensitive | Blocked by Some |
| | | | | Overlapping |
| | | | | Combinations |
| ClaI | AT/CGAT | Blocked by | Not Sensitive | Blocked |
| | | Overlapping | | |
| | | Methylation | | |
| CspCI | (11/13)CAANNNNNGTGG(12/10) | Not Sensitive | Not Sensitive | Not Sensitive |
| | (SEQ ID NO: 5) | | | |
| CviAII | C/ATG | Not Sensitive | Not Sensitive | Not Sensitive |
| CviKI-1 | RG/CY | Not Sensitive | Not Sensitive | Not Sensitive |
| CviQI | G/TAC | Not Sensitive | Not Sensitive | Not Sensitive |
| DdeI | C/TNAG | Not Sensitive | Not Sensitive | Not Sensitive |
| DpnI | GA/TC | Not Sensitive | Not Sensitive | Blocked by |
| | | | | Overlapping |
| | | | | Methylation |
| DpnII | /GATC | Blocked | Not Sensitive | Not Sensitive |
| DraI | TTT/AAA | Not Sensitive | Not Sensitive | Not Sensitive |
| DraIII | CACNNN/GTG | Not Sensitive | Not Sensitive | Impaired by |
| | | | | Overlapping |
| | | | | Methylation |
| DrdI | GACNNNN/NNGTC | Not Sensitive | Not Sensitive | Blocked by |
| | | | | Some |
| | | | | Overlapping |
| | | | | Combinations |
| EaeI | Y/GGCCR | Not Sensitive | Blocked by | Blocked by |
| | | | Overlapping | Overlapping |
| | | | Methylation | Methylation |
| EagI | C/GGCCG | Not Sensitive | Not Sensitive | Blocked |
| EagI-HF ™ | C/GGCCG | Not Sensitive | Not Sensitive | Blocked |
| EarI | CTCTTC(1/4) | Not Sensitive | Not Sensitive | Impaired by |
| | | | | Overlapping |
| | | | | Methylation |
| EciI | GGCGGA(11/9) | Not Sensitive | Not Sensitive | Blocked by Some |
| | | | | Overlapping |
| | | | | Combinations |
| Eco53kI | GAG/CTC | — | — | — |
| EcoNI | CCTNN/NNNAGG | Not Sensitive | Not Sensitive | Not Sensitive |
| EcoO109I | RG/GNCCY | Not Sensitive | Blocked by | Not Sensitive |
| | | | Overlapping | |
| | | | Methylation | |
| EcoP15I | CAGCAG(25/27) | Not Sensitive | Not Sensitive | Not Sensitive |
| EcoRI | G/AATTC | Not Sensitive | Not Sensitive | Blocked by Some |
| | | | | Overlapping |
| | | | | Combinations |
| EcoRI- | G/AATTC | Not Sensitive | Not Sensitive | Blocked by Some |
| HF ™ | | | | Overlapping |
| | | | | Combinations |
| EcoRV | GAT/ATC | Not Sensitive | Not Sensitive | Impaired by Some |
| | | | | Overlapping |
| | | | | Combinations |
| EcoRV- | GAT/ATC | Not Sensitive | Not Sensitive | Impaired by Some |
| HF ™ | | | | Overlapping |
| | | | | Combinations |
| FatI | /CATG | Not Sensitive | Not Sensitive | Not Sensitive |
| FauI | CCCGC(4/6) | Not Sensitive | Not Sensitive | Blocked |
| Fnu4HI | GC/NGC | Not Sensitive | Not Sensitive | Blocked by |
| | | | | Overlapping |
| | | | | Methylation |
| FokI | GGATG(9/13) | Not Sensitive | Impaired by | Impaired by |
| | | | Overlapping | Overlapping |
| | | | Methylation | Methylation |
| FseI | GGCCGG/CC | Not Sensitive | Impaired by | Blocked |
| | | | Some Over- | |
| | | | lapping | |
| | | | Combinations | |
| FspI | TGC/GCA | Not Sensitive | Not Sensitive | Blocked |
| HaeII | RGCGC/Y | Not Sensitive | Not Sensitive | Blocked |
| HaeIII | GG/CC | Not Sensitive | Not Sensitive | Not Sensitive |
| HgaI | GACGC(5/10) | Not Sensitive | Not Sensitive | Blocked |
| HhaI | GCG/C | Not Sensitive | Not Sensitive | Blocked |
| HincII | GTY/RAC | Not Sensitive | Not Sensitive | Blocked by Some |
| | | | | Overlapping |
| | | | | Combinations |
| HindIII | A/AGCTT | Not Sensitive | Not Sensitive | Not Sensitive |
| HinfI | G/ANTC | Not Sensitive | Not Sensitive | Blocked by Some |
| | | | | Overlapping |
| | | | | Combinations |
| HinP1I | G/CGC | Not Sensitive | Not Sensitive | Blocked |
| HpaI | GTT/AAC | Not Sensitive | Not Sensitive | Blocked by Some |
| | | | | Overlapping |
| | | | | Combinations |
| HpaII | C/CGG | Not Sensitive | Not Sensitive | Blocked |
| HphI | GGTGA(8/7) | Blocked by | Not Sensitive | Not Sensitive |
| | | Overlapping | | |
| | | Methylation | | |
| Hpy166II | GTN/NAC | Not Sensitive | Not Sensitive | Blocked by |
| | | | | Overlapping |
| | | | | Methylation |
| Hpy188I | TCN/GA | Blocked by | Not Sensitive | Not Sensitive |
| | | Overlapping | | |
| | | Methylation | | |
| Hpy188III | TC/NNGA | Blocked by | Not Sensitive | Blocked by |
| | | Overlapping | | Overlapping |
| | | Methylation | | Methylation |
| Hpy99I | CGWCG/ | Not Sensitive | Not Sensitive | Blocked |
| HpyAV | CCTTC(6/5) | Not Sensitive | Not Sensitive | Impaired by |
| | | | | Overlapping |
| | | | | Methylation |
| HpyCH4III | ACN/GT | Not Sensitive | Not Sensitive | Not Sensitive |
| HpyCH4IV | A/CGT | Not Sensitive | Not Sensitive | Blocked |
| HpyCH4V | TG/CA | Not Sensitive | Not Sensitive | Not Sensitive |
| I-CeuI | CGTAACTATAACGGTCCTAAGGTAGCGAA | — | — | — |
| | (−9/−13) (SEQ ID NO: 6) | | | |
| I-SceI | TAGGGATAACAGGGTAAT(−9/−13) | — | — | — |
| | (SEQ ID NO: 7) | | | |
| KasI | G/GCGCC | Not Sensitive | Not Sensitive | Blocked |
| KpnI | GGTAC/C | Not Sensitive | Not Sensitive | Not Sensitive |
| KpnI-HF ™ | GGTAC/C | — | — | — |
| MboI | /GATC | Blocked | Not Sensitive | Impaired by |
| | | | | Overlapping |
| | | | | Methylation |
| MboII | GAAGA(8/7) | Blocked by | Not Sensitive | Not Sensitive |
| | | Overlapping | | |
| | | Methylation | | |
| MfeI | C/AATTG | Not Sensitive | Not Sensitive | Not Sensitive |
| MfeI-HF ™ | C/AATTG | Not Sensitive | Not Sensitive | Not Sensitive |
| MluI | A/CGCGT | Not Sensitive | Not Sensitive | Blocked |
| MlyI | GAGTC(5/5) | Not Sensitive | Not Sensitive | |
| MmeI | TCCRAC(20/18) | Not Sensitive | Not Sensitive | Blocked by |
| | | | | Overlapping |
| | | | | Methylation |
| MnII | CCTC(7/6) | Not Sensitive | Not Sensitive | Not Sensitive |
| MscI | TGG/CCA | Not Sensitive | Blocked by | Not Sensitive |
| | | | Overlapping | |
| | | | Methylation | |
| MseI | T/TAA | Not Sensitive | Not Sensitive | Not Sensitive |
| MslI | CAYNN/NNRTG | Not Sensitive | Not Sensitive | Not Sensitive |
| MspA1I | CMG/CKG | Not Sensitive | Not Sensitive | Blocked by |
| | | | | Overlapping |
| | | | | Methylation |
| MspI | C/CGG | Not Sensitive | Not Sensitive | Not Sensitive |
| MwoI | GCNNNNN/NNGC | Not Sensitive | Not Sensitive | Blocked by Some |
| | | | | Overlapping |
| | | | | Combinations |
| NaeI | GCC/GGC | Not Sensitive | Not Sensitive | Blocked |
| NarI | GG/CGCC | Not Sensitive | Not Sensitive | Blocked |
| Nb.BbvCI | CCTCAGC | Not Sensitive | Not Sensitive | Not Sensitive |
| Nb.BsmI | GAATGC | Not Sensitive | Not Sensitive | Not Sensitive |
| Nb.BsrDI | GCAATG | Not Sensitive | Not Sensitive | Not Sensitive |
| Nb.BtsI | GCAGTG | — | — | — |
| NciI | CC/SGG | Not Sensitive | Not Sensitive | Impaired by |
| | | | | Overlapping |
| | | | | Methylation |
| NcoI | C/CATGG | Not Sensitive | Not Sensitive | Not Sensitive |
| NcoI-HF ™ | C/CATGG | Not Sensitive | Not Sensitive | Not Sensitive |
| NdeI | CA/TATG | Not Sensitive | Not Sensitive | Not Sensitive |
| NgoMIV | G/CCGGC | Not Sensitive | Not Sensitive | Blocked |
| NheI | G/CTAGC | Not Sensitive | Not Sensitive | Blocked by Some |
| | | | | Overlapping |
| | | | | Combinations |
| NheI-HF ™ | G/CTAGC | Not Sensitive | Not Sensitive | Blocked by Some |
| | | | | Overlapping |
| | | | | Combinations |
| NlaIII | CATG/ | Not Sensitive | Not Sensitive | Not Sensitive |
| NlaIV | GGN/NCC | Not Sensitive | Blocked by | Blocked by |
| | | | Overlapping | Overlapping |
| | | | Methylation | Methylation |
| NmeAIII | GCCGAG(21/19) | Not Sensitive | Not Sensitive | Not Sensitive |
| NotI | GC/GGCCGC | Not Sensitive | Not Sensitive | Blocked |
| NotI-HF ™ | GC/GGCCGC | Not Sensitive | Not Sensitive | Blocked |
| NruI | TCG/CGA | Blocked by | Not Sensitive | Blocked |
| | | Overlapping | | |
| | | Methylation | | |
| NsiI | ATGCA/T | Not Sensitive | Not Sensitive | Not Sensitive |
| NspI | RCATG/Y | Not Sensitive | Not Sensitive | Not Sensitive |
| Nt.AlwI | GGATC(4/−5) | Blocked | Not Sensitive | Not Sensitive |
| Nt.BbvCI | CCTCAGC(-5/-7) | Not Sensitive | Not Sensitive | Blocked by Some |
| | | | | Overlapping |
| | | | | Combinations |
| Nt.BsmAI | GTCTC(1/−5) | Not Sensitive | Not Sensitive | Blocked |
| Nt.BspQI | GCTCTTC(1/−7) | Not Sensitive | Not Sensitive | Not Sensitive |
| Nt.BstNBI | GAGTC(4/−5) | Not Sensitive | Not Sensitive | Not Sensitive |
| Nt.CviPII | (0/−1)CCD | Not Sensitive | Not Sensitive | Blocked |
| PacI | TTAAT/TAA | Not Sensitive | Not Sensitive | Not Sensitive |
| PaeR7I | C/TCGAG | Not Sensitive | Not Sensitive | Blocked |
| PciI | A/CATGT | Not Sensitive | Not Sensitive | Not Sensitive |
| PfIFI | GACN/NNGTC | Not Sensitive | Not Sensitive | Not Sensitive |
| PfIMI | CCANNNN/NTGG | Not Sensitive | Blocked by | Not Sensitive |
| | | | Overlapping | |
| | | | Methylation | |
| PhoI | GG/CC | Not Sensitive | Impaired by | Impaired by Some |
| | | | Some Over- | Overlapping |
| | | | lapping | |
| | | | Combinations | Combinations |
| PI-PspI | TGGCAAACAGCTATTATGGGTATTATGGGT | — | — | — |
| | (−13/−17) (SEQ ID NO: 8) | | | |
| PI-SceI | ATCTATGTCGGGTGCGGAGAAAGAGGTAAT | — | — | — |
| | (−15/−19) (SEQ ID NO: 9) | | | |
| PleI | GAGTC(4/5) | Not Sensitive | Not Sensitive | Blocked by Some |
| | | | | Overlapping |
| | | | | Combinations |
| PmeI | GTTT/AAAC | Not Sensitive | Not Sensitive | Blocked by Some |
| | | | | Overlapping |
| | | | | Combinations |
| PmlI | CAC/GTG | Not Sensitive | Not Sensitive | Blocked |
| PpuMI | RG/GWCCY | Not Sensitive | Blocked by | Not Sensitive |
| | | | Overlapping | |
| | | | Methylation | |
| PshAI | GACNN/NNGTC | Not Sensitive | Not Sensitive | Blocked by Some |
| | | | | Overlapping |
| | | | | Combinations |
| PsiI | TTA/TAA | Not Sensitive | Not Sensitive | Not Sensitive |
| PspGI | /CCWGG | Not Sensitive | Blocked | Not Sensitive |
| PspOMI | G/GGCCC | Not Sensitive | Blocked by | Blocked by |
| | | | Overlapping | Overlapping |
| | | | Methylation | Methylation |
| PspXI | VC/TCGAGB | Not Sensitive | Not Sensitive | Impaired |
| PstI | CTGCA/G | Not Sensitive | Not Sensitive | Not Sensitive |
| PstI-HF ™ | CTGCA/G | — | — | — |
| PvuI | CGAT/CG | Not Sensitive | Not Sensitive | Blocked |
| PvuII | CAG/CTG | Not Sensitive | Not Sensitive | Not Sensitive |
| PvuII- | CAG/CTG | Not Sensitive | Not Sensitive | Not Sensitive |
| HF ™ | | | | |
| RsaI | GT/AC | Not Sensitive | Not Sensitive | Blocked by Some |
| | | | | Overlapping |
| | | | | Combinations |
| RsrII | CG/GWCCG | Not Sensitive | Not Sensitive | Blocked |
| SacI | GAGCT/C | Not Sensitive | Not Sensitive | Not Sensitive |
| SacI- | GAGCT/C | Not Sensitive | Not Sensitive | Not Sensitive |
| HF ™ | | | | |
| SacII | CCGC/GG | Not Sensitive | Not Sensitive | Blocked |
| SaII | G/TCGAC | Not Sensitive | Not Sensitive | Blocked |
| SaII- | G/TCGAC | Not Sensitive | Not Sensitive | Blocked |
| HF ™ | | | | |
| SapI | GCTCTTC(1/4) | Not Sensitive | Not Sensitive | Not Sensitive |
| Sau3AI | /GATC | Not Sensitive | Not Sensitive | Blocked by |
| | | | | Overlapping |
| | | | | Methylation |
| Sau96I | G/GNCC | Not Sensitive | Blocked by | Blocked by |
| | | | Overlapping | Overlapping |
| | | | Methylation | Methylation |
| SbfI | CCTGCA/GG | Not Sensitive | Not Sensitive | Not Sensitive |
| SbfI- | CCTGCA/GG | Not Sensitive | Not Sensitive | Not Sensitive |
| HF ™ | | | | |
| ScaI | AGT/ACT | Not Sensitive | Not Sensitive | Not Sensitive |
| ScaI- | AGT/ACT | Not Sensitive | Not Sensitive | Not Sensitive |
| HF ™ | | | | |
| ScrFI | CC/NGG | Not Sensitive | Blocked by | Blocked by |
| | | | Overlapping | Overlapping |
| | | | Methylation | Methylation |
| SexAI | A/CCWGGT | Not Sensitive | Blocked | Not Sensitive |
| SfaNI | GCATC(5/9) | Not Sensitive | Not Sensitive | Impaired by Some |
| | | | | Overlapping |
| | | | | Combinations |
| SfcI | C/TRYAG | Not Sensitive | Not Sensitive | Not Sensitive |
| SfiI | GGCCNNNN/NGGCC | Not Sensitive | Impaired by | Blocked by Some |
| | | | Overlapping | Overlapping |
| | | | Methylation | Combinations |
| SfoI | GGC/GCC | Not Sensitive | Blocked by | Blocked |
| | | | Some Over- | |
| | | | lapping | |
| | | | Combinations | |
| SgrAI | CR/CCGGYG | Not Sensitive | Not Sensitive | Blocked |
| SmaI | CCC/GGG | Not Sensitive | Not Sensitive | Blocked |
| SmlI | C/TYRAG | Not Sensitive | Not Sensitive | Not Sensitive |
| SnaBI | TAC/GTA | Not Sensitive | Not Sensitive | Blocked |
| SpeI | A/CTAGT | Not Sensitive | Not Sensitive | Not Sensitive |
| SphI | GCATG/C | Not Sensitive | Not Sensitive | Not Sensitive |
| SphI- | GCATG/C | Not Sensitive | Not Sensitive | Not Sensitive |
| HF ™ | | | | |
| SspI | AAT/ATT | Not Sensitive | Not Sensitive | Not Sensitive |
| SspI- | AAT/ATT | Not Sensitive | Not Sensitive | Not Sensitive |
| HF ™ | | | | |
| StuI | AGG/CCT | Not Sensitive | Blocked by | Not Sensitive |
| | | | Overlapping | |
| | | | Methylation | |
| StyD4I | /CCNGG | Not Sensitive | Blocked by | Impaired by |
| | | | Overlapping | Overlapping |
| | | | Methylation | Methylation |
| StyI | C/CWWGG | Not Sensitive | Not Sensitive | Not Sensitive |
| StyI- | C/CWWGG | — | — | — |
| HF ™ | | | | |
| SwaI | ATTT/AAAT | Not Sensitive | Not Sensitive | Not Sensitive |
| TaqαI | T/CGA | Blocked by | Not Sensitive | Not Sensitive |
| | | Overlapping | | |
| | | Methylation | | |
| TfiI | G/AWTC | Not Sensitive | Not Sensitive | Blocked by Some |
| | | | | Overlapping |
| | | | | Combinations |
| TliI | C/TCGAG | Not Sensitive | Not Sensitive | Impaired |
| TseI | G/CWGC | Not Sensitive | Not Sensitive | Blocked by Some |
| | | | | Overlapping |
| | | | | Combinations |
| Tsp45I | /GTSAC | Not Sensitive | Not Sensitive | Not Sensitive |
| Tsp509I | /AATT | Not Sensitive | Not Sensitive | Not Sensitive |
| TspMI | C/CCGGG | Not Sensitive | Not Sensitive | Blocked |
| TspRI | NNCASTGNN/ | Not Sensitive | Not Sensitive | Not Sensitive |
| Tth111I | GACN/NNGTC | Not Sensitive | Not Sensitive | Not Sensitive |
| XbaI | T/CTAGA | Blocked by | Not Sensitive | Not Sensitive |
| | | Overlapping | | |
| | | Methylation | | |
| XcmI | CCANNNNN/NNNNTGG | Not Sensitive | Not Sensitive | Not Sensitive |
| XhoI | C/TCGAG | Not Sensitive | Not Sensitive | Impaired |
| XmaI | C/CCGGG | Not Sensitive | Not Sensitive | Impaired |
| XmnI | GAANN/NNTTC | Not Sensitive | Not Sensitive | Not Sensitive |
| ZraI | GAC/GTC | Not Sensitive | Not Sensitive | Blocked |

### VI. THERAPEUTICS

Embodiments contemplate the treatment of a patient with FSHD comprising administering a pharmaceutical composition comprising a compound that provides SMCHD1 activity to the patient. In some embodiments, the SMCHD1 protein may be used to treat a patient with FSHD. The methods involves administering to the patients a pharmaceutical composition comprising a polypeptide that is at least of 70%, 75%, 80%, 85%, 90%, 95%, 96%, 97%, 98%, or 99% or higher sequence identity, including all values and ranges there between, or any range derivable therein, compared to a SMCHD1 protein (Genebank accession number: NP_{—}56110) using the methods described herein (e.g., BLAST analysis using standard parameters).

In certain embodiments, a vector comprising a promoter operably linked to a nucleic acid segment encoding a polypeptide that is at least of 70%, 75%, 80%, 85%, 90%, 95%, 96%, 97%, 98%, or 99% or higher sequence identity, including all values and ranges there between, or any range derivable therein, compared to a SMCHD1 protein. In additional embodiments, the nucleic acid segment is of at least of 70%, 75%, 80%, 85%, 90%, 95%, 96%, 97%, 98%, or 99% or higher sequence identity, including all values and ranges there between, or any range derivable therein, compared to SEQ ID NO:1 using the methods described herein (e.g., BLAST analysis using standard parameters).

Other embodiments related to treatment may concern the DUX4 region. Examples are provided in PCT/US2011/048318, U.S. Application Ser. Nos. 61/374,967, 61/384,609, 61/513,456, 61/513,467, which are all incorporated by reference. Treatments include, but are not limited to, administration of an agent capable of inhibiting or suppressing the level of DUC4-fl expression or an agent capable of inhibiting DUC4-fl mediated transcription activation. In certain embodiments, the treatment concerns an agent that enhances nonsense mediated decay and enhances the drgration of DUX4mRNA. In further embodiments, the agent comprises a DUC4-s polypeptide or a nucleic acid molecule encoding all or part of a DUC4-s polypeptide.

The nucleic acid segments used herein, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed. The total length may be limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy.

The pharmaceutical compositions contemplated herein comprise an effective amount of one or more compound capable of providing SMCHD1 activity to cells of FSHD patients or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one compound described herein or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical composition described herein may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The pharmaceutical composition describe herein may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

In further embodiments, the pharmaceutical composition suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The composition is combined with the carrier in any convenient and practical manner, i.e., by solution, suspension, emulsification, admixture, encapsulation, absorption and the like. Such procedures are routine for those skilled in the art.

In a specific embodiment, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

Further embodiments concern the use of a pharmaceutical lipid vehicle compositions that include polypeptide or vector or other compound described herein, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the composition disclosed herein may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition administered to a patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In some embodiments, the pharmaceutical compositions contemplated herein are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the compositions described herein may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In additional embodiments, the pharmaceutical composition described herein may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,753,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In further embodiments, the composition disclosed herein may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

The therapeutic methods for treating FSHDs by utilizing the therapeutic agents capable of blocking post-translational modification of SMCHD1 protein are also contemplated. The post-translational modification of SMCHD1 protein can be any post-translational modifications which lead to or increase SMCHD1 protein degradation, such as, but not limited to, phosphorylation or sumolyation. The therapeutic methods can also be used in combination with other treatment methods for FSHDs.

In additional aspects, the therapeutics methods for treating a disease associated with excessive SMCHD1 activity, such as increased SMCHD1 activity, increased SMCHD1 expression, are also contemplated. The small interference RNA, SMCHD1 inhibitor, and other agents capable of deceasing SMCHD1 activity can be employed to treat a disease with excessive SMCHD1 activity.

It is additionally contemplated that the pharmaceutical composition described herein may be used to treat other diseases associated with the epigenetic repression of a genomic region being modified by a SMCHD1 variant as described herein, such as Fragile X syndrome or cancers associated with epigenetic silencing of tumor suppressor genes.

### VII. KITS

The embodiments additionally provide kits for detecting FSHD in a subject. The contents of the kits may comprise one or more agents for detecting the presence of a variant of SMCHD1 as disclosed herein, detecting reduced SMCHD1 mRNA expression, detecting reduced SMCHD1 protein expression, or detecting reduced binding between SMCHD1 and D4Z4 arrays or a combination thereof. The kits may comprise one or more enzymes, such as polymerase, restriction enzyme, ligase, one or more probes, nucleotides, labels, labeling reagent, one or more buffers or any additional agent required for achieving the method described herein.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

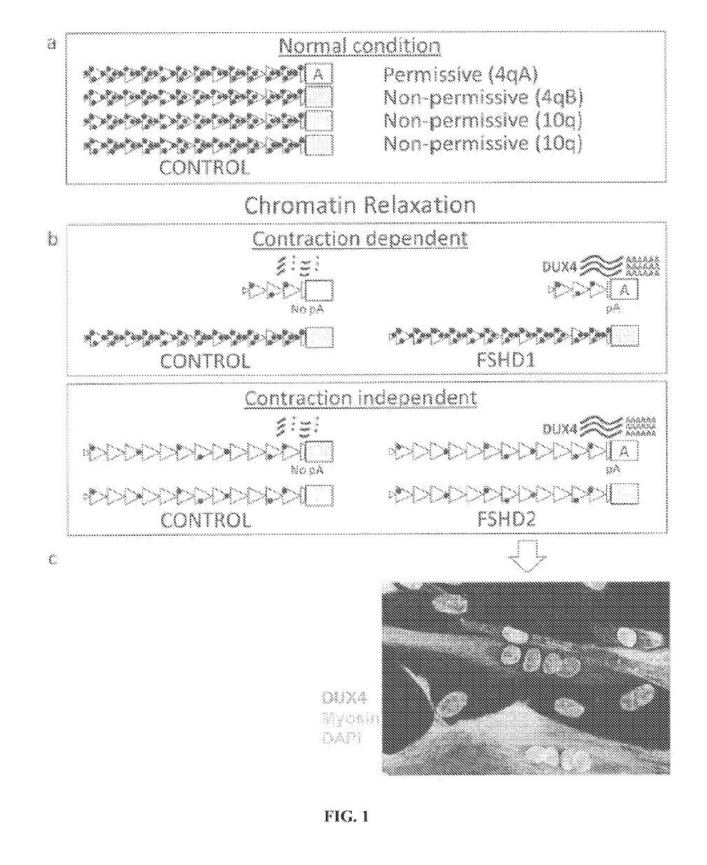

FSHD is clinically characterized by variable penetrance and often asymmetric presentation of facial and upper-extremity muscle weakness (Statland and Tawil, 2011) FSHD1 and FSHD2 are phenotypically indistinguishable and are both associated with DNA hypomethylation and decreased repressive heterochromatin of the D4Z4 array, which are collectively referred to as chromatin relaxation (Balog et al., 2012; Bodega et al., 2009; Cabianca et al., 2012; de Greef et al., 2009; Jiang et al., 2003; van Overveld et al., 2003; Zeng et al., 2009) (FIG. 1). Each D4Z4 unit contains a copy of the DUX4 retrogene (Gabriels et al., 1999; Hewitt et al., 1994; Lyle et al., 1995; Snider et al., 2009; Snider et al., 2010), and this relaxation lifts the somatic repression of DUX4 leading to a variegated pattern of DUX4 protein expression in a subset of skeletal muscle nuclei (van der Maarel et al., 2011). Chromatin relaxation in FSHD1 is associated with a contraction of the array to <10 D4Z4 repeat units and therefore has a dominant inheritance pattern linked to the contracted array. In FSHD2, chromatin relaxation is independent of the size of the D4Z4 array and occurs on both chromosome-4 D4Z4 arrays and also on the highly homologous arrays on chromosome 10 (Balog et al., 2012; van Overveld et al., 2003; Zeng et al., 2009; van Deutekom et al., 1993; Wijmenga et al., 1992).

D4Z4 chromatin relaxation must occur on a specific chromosome-4 haplotype in order to cause FSHD1 and FSHD2. This haplotype contains a polyadenylation (pA) signal to stabilize DUX4 mRNA in skeletal muscle (Snider et al., 2010; Dixit et al., 2007; Lemmers et al., 2002; Lemmers et al., 2010; Spurlock et al., 2010; Thomas et al., 2007). Chromosomes 4 and 10 that lack this pA signal fail to produce DUX4 protein; consequently, D4Z4 chromatin relaxation and transcriptional derepression on these non-permissive haplotypes do not lead to disease. Because chromatin relaxation occurs at D4Z4 repeats in FSHD2, the inventors sought to determine whether an inherited defect in a modifier of D4Z4 repeat-mediated epigenetic repression might cause FSHD2 when combined with an FSHD-permissive DUX4 allele.

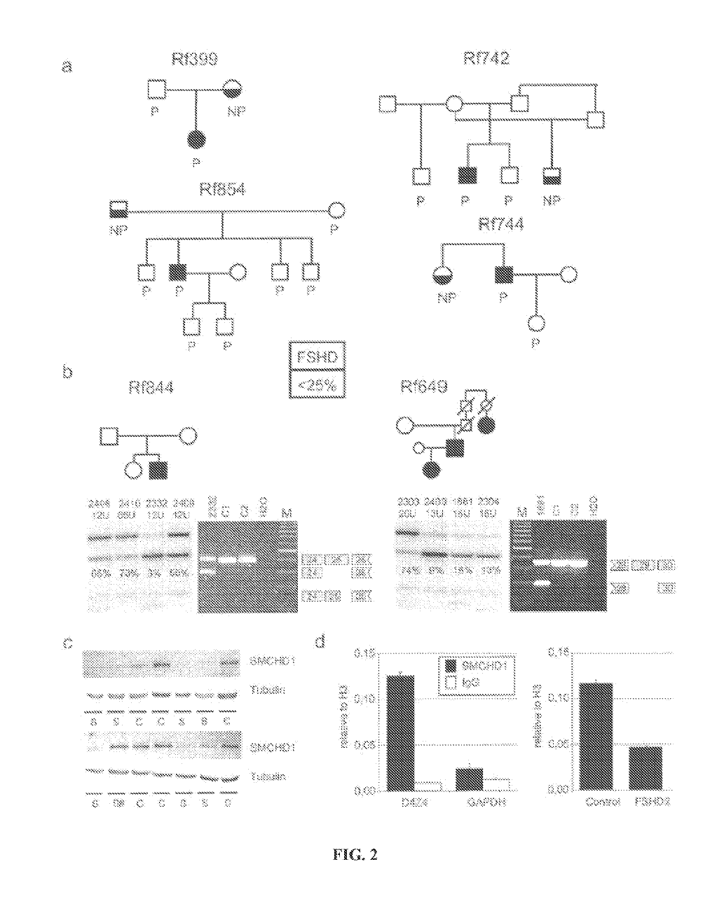

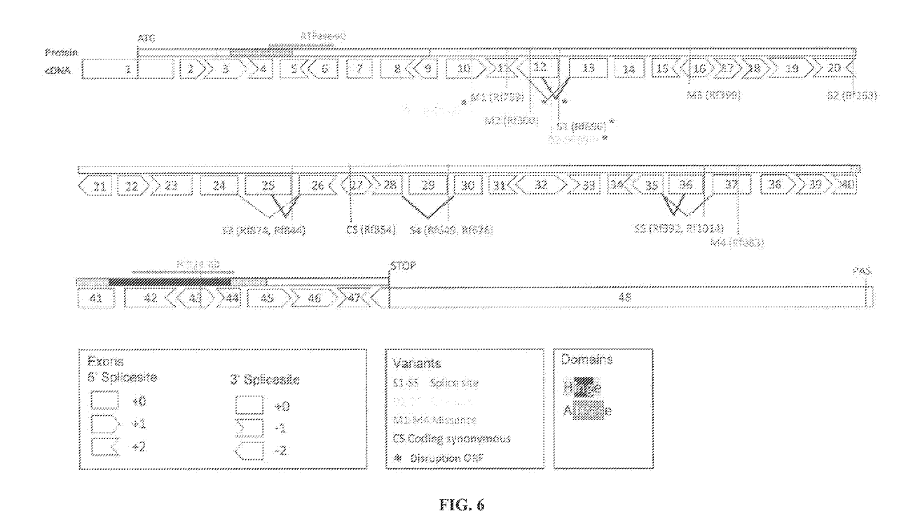

In kindreds identified by an FSHD2 proband, the inventors discovered that D4Z4 hypomethylation segregated as a dominant genetic trait that was not linked to the chromosome-4 or -10 D4Z4 array haplotypes (FIG. 2, Materials and Methods below, and FIGS. 4-5). In these kindreds, FSHD2 individuals inherited both the hypomethylation trait and the FSHD-permissive chromosome-4 haplotype with the DUX4 pA signal, suggesting that two independently segregating loci cause and determine the penetrance of FSHD2.

To identify the locus controlling the D4Z4 hypomethylation trait, the inventors performed whole exome sequencing (Bamshad et al., 2011) of twelve individuals in seven unrelated FSHD2 families: five with dominant segregation of the hypomethylation trait and two with sporadic hypomethylation and FSHD2 (FIG. 5). Families were stratified according to the criteria listed in Table 1 and described in Materials and Methods.

TABLE 1
Criteria used to prioritize families for WES

| Criteria | Number of families |
|---|---|
| FseI Methylation <25% | 41 |
| Both chromosome 4q D4Z4 arrays >10 units | 40 |
| Not more than one chromosome 10q | 39 |
| D4Z4 array <11 units | |
| Inheritance Pattern: | |
| Dominant inheritance | 13 |
| De novo D4Z4 hypomethylation | 7 |
| Unknown (not informative) | 19 |

Maximum D4Z4 methylation at FseI site in patients was set at 25%. The inventors excluded families in which one of the individuals with D4Z4 methylation <25% had a D4Z4 repeat array of <10 units on a permissive allele or more than one array of <10 units. Families were further categorized according to the inheritance pattern of D4Z4 hypomethylation.

The inventors identified rare and potentially pathogenic variants in the SMCHD1 (Structural maintenance of chromosomes flexible hinge domain-containing 1) gene in all individuals with D4Z4 hypomethylation with the exception of members of one family, Rf854 (Table 2). These variants were not present in public (dbSNP132 and the 1000 Genomes Project) or internal databases or in family members with normal D4Z4 methylation levels.

TABLE 2
SMCHD1 variants identified

| Family | | | | | | |
|---|---|---|---|---|---|---|
| analysis | Inheritance | Mutation type | Nr | Position¹ | Mutation position² | RNA |
| Rf1033 | Unknown | deletion | D1 | exon 10 | g.2697999_2698003del | WT + mutant |
| | | | | | | trancript* |
| Rf739 | De novo | missense | M1 | exon 11 | g.2700705G>C | WT + mutant |
| | | | | | | transcript |
| Rf300 | De novo | missense | M2 | exon 12 | g.2700743T>C | WT + mutant |
| | | | | | | transcript |
| Rf393 | Paternal | deletion | D2 | exon 12 | g.2700875_2700875del | WT + mutant |
| | | | | | | transcript* |
| Rf696 | Unknown | 5′ splice site | S1 | intron 12 | g.2701019A>G | WT + skip |
| | | | | | | exon 12* + |
| | | | | | | cryptic |
| | | | | | | splicing 12* |
| Rf399 | Maternal | missense | M3 | exon 16 | g.2707565C>T | WT + mutant |
| | | | | | | transcript |
| Rf268 | Unknown | 5′ splice site | S2 | exon 20 | g.2722661G>A | ** |
| Rf844 | De novo | 5′ splice site | S3 | intron 25 | g.2732488_2732492del | WT + exon |
| | | | | | | 25 skip + |
| | | | | | | cryptic |
| | | | | | | splicing 25 |
| Rf874 | Maternal | 5′ splice site | S3 | intron 25 | g.2732488_2732492del | ** |
| Rf854 | Paternal | coding synonymous³ | CS | exon 27 | g.2739448T>A | WT + mutant |
| | | | | | | transcript |
| Rf649 | Paternal | 5′ splice site | S4 | intron 29 | g.2743927G>A | WT + cryptic |
| | | | | | | splicing |
| Rf676 | Unknown | 5′ splice site | S4 | intron 29 | g.2743927G>A | ** |
| Rf1014 | Paternal | 5′ splice site | S5 | exon 36 | g.2762234G>A | WT + exon |
| | | | | | | 36 skip |
| Rf392 | Maternal | 5′ splice site | S5 | exon 36 | g.2762234G>A | WT + exon |
| | | | | | | 36 skip + |
| | | | | | | cryptic |
| | | | | | | splicing 36 |
| Rf683 | Unknown | missence | M4 | exon 37 | g.2763729T>C | WT + mutant |
| | | | | | | transcript |

¹Exon number is based on transcript ENST00000320876
²Genomic position is based on hg19
³Present at frequency 0.0055 in 1000 Genomes
*Disruption open reading frame
** No RNA available

Column 4 of Table 2 shows the position of the mutation according to FIG. 13. The position of the mutation in the SMCHD1 gene is given as well as a summary of the RNA analysis.

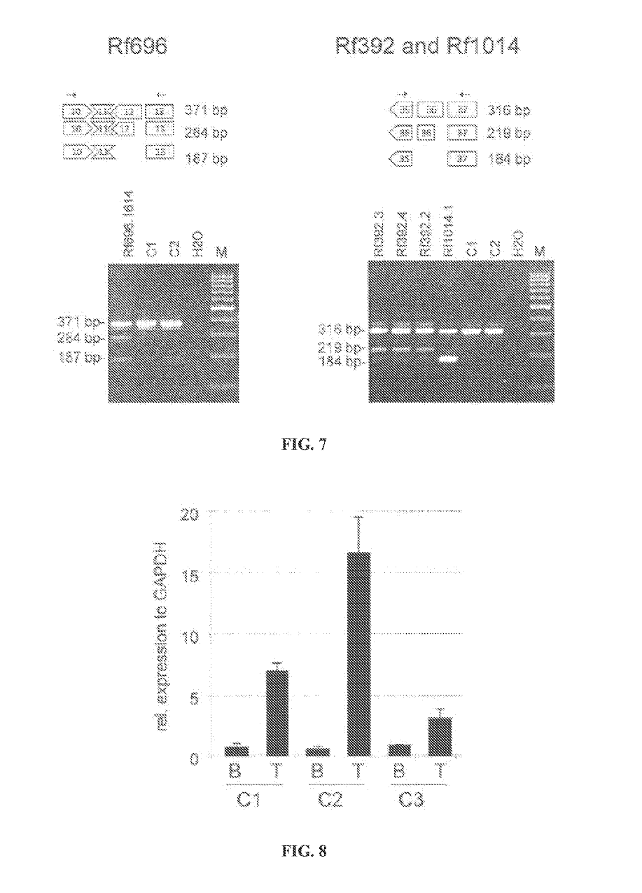

The inventors confirmed the presence of these variants by Sanger sequencing and included 16 additional unrelated FSHD2 families for which DNA or RNA was available. The inventors identified heterozygous out-of-frame deletions, splice-site variants, and heterozygous missense variants in SMCHD1 in 14/23 (61%) families (Table 2 and FIG. 6). The inventors also confirmed that the splice-site variants altered the normal SMCHD1 mRNA by exclusion of exons and cryptic splice site usage (FIG. 2b and FIG. 7).

Because heterozygous SMCHD1 variants co-segregated with D4Z4 hypomethylation in FSHD2 families or occurred de novo in sporadic hypomethylation/FSHD2 individuals (FIG. 2b and FIG. 5), SMCHD1 haploinsufficiency was considered as a candidate disease mechanism, particularly since many of the variants were predicted to affect the production of the full protein. Indeed, fibroblasts from FSHD2 patients with non-synonymous or splice-site variants in SMCHD1 had substantially reduced SMCHD1 protein levels (FIG. 2c). The inventors found normal levels of SMCHD1 protein in the hypomethylated FSHD2 individual in family RP854 that did not have an SMCHD/mutation (FIG. 2c), suggesting that FSHD2 in this family has a genetic cause other than SMCHD1 haploinsufficiency. Finally, chromatin immunoprecipitation (ChIP) demonstrated the presence of SMCHD1 on the D4Z4 array and reduced levels of this association in FSHD2 individuals with SMCHD1 variants (FIG. 2d). Together, these results support haploinsufficiency of SMCHD1 as a cause of D4Z4 hypomethylation in unrelated FSHD2 kindreds.

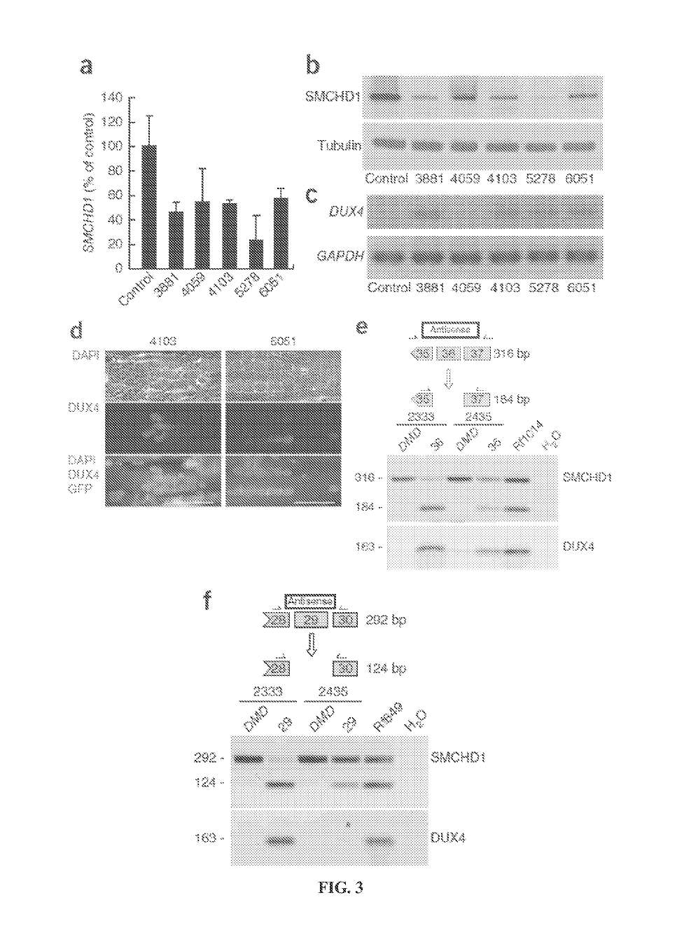

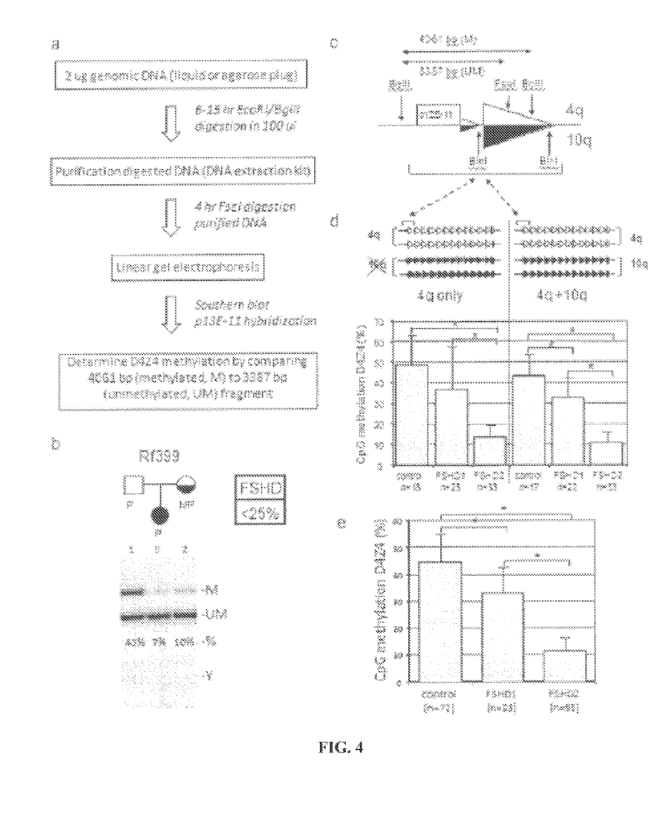

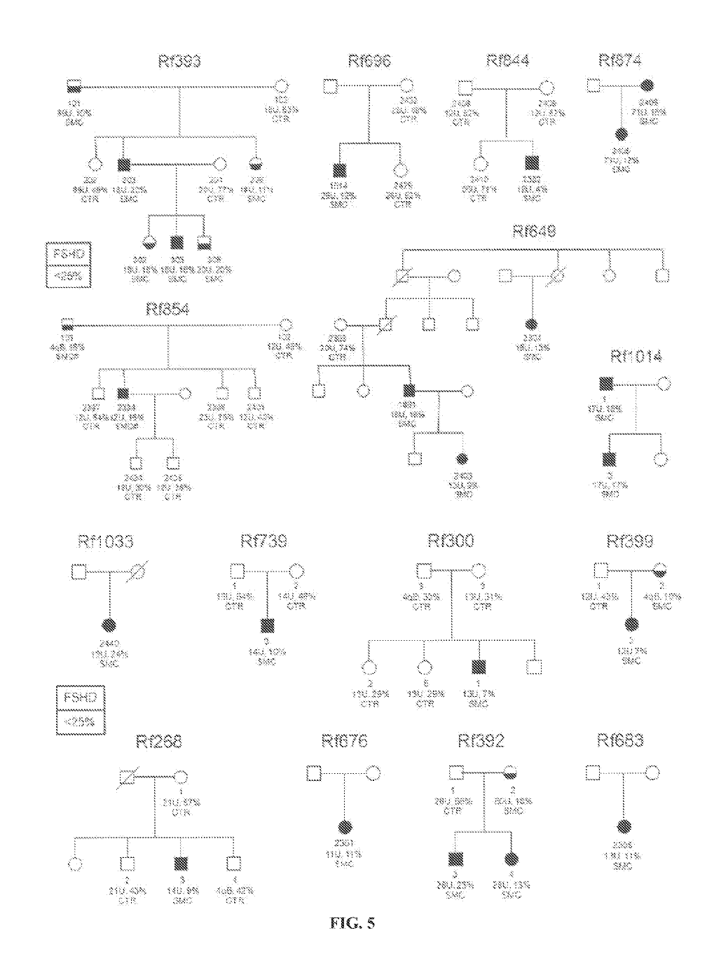

FSHD is characterized by low-level variegated expression of DUX4 in skeletal muscle. Therefore, the inventors assessed DUX4 expression in skeletal muscle cells from control individuals after decreasing SMCHD1 by RNA interference (FIGS. 3a-b). The inventors detected no DUX4 mRNA in primary myotubes from an unaffected individual with a normal-sized and methylated D4Z4 array on the FSHD-permissive DUX4 pA haplotype. In contrast, DUX4 was transcriptionally activated in these myotubes (FIG. 3c) when SMCHD1 transcripts and protein were suppressed to <50% of normal levels. The inventors observed a variegated pattern of DUX4 protein in myotubes in all samples with adequate SMCHD1 knockdown (FIG. 3d); this pattern is similar to that seen in myotubes from FSHD2 patients (FIG. 1c). Cells expressing a scrambled or ineffective shRNA did not express DUX4 (FIG. 3), indicating that the re-expression of DUX4 was caused by the decreased levels of SMCHD1 and not a non-specific consequence of introducing any shRNA sequence into a cell.

To demonstrate that the SMCHD1 splice variants identified in FSHD2 patients result in DUX4 expression, the inventors manipulated SMCHD1 pre-mRNA splicing in skeletal muscle cells using antisense oligonucleotides (AONs) directed to exon 29 or 36. These AONs induced skipping of SMCHD1 exon 29 or 36 at rates comparable to those detected in some FSHD2 patients (FIG. 3e) and resulted in transcription of DUX4 (FIG. 3e). Thus, SMCHD1 activity is necessary for the somatic repression of DUX4, and reduction of this activity produces D4Z4 arrays that express DUX4 when an FSHD-permissive DUX4 haplotype is present, with a pattern of variegated expression similar to that observed in FSHD1 and FSHD2 myotube cultures.

SMCHD1 belongs to the SMC gene superfamily that regulates chromatin repression of loci in many different organisms, including silencing mating loci in yeast (Bhalla et al., 2002), dosage compensation in C. elegans (Lieb et al., 1996; Chuang et al., 1994), position-effect variegation in D. melanogaster (Dej et al., 2004), and RNA-directed DNA methylation in Arabidopsis (Kanno et al., 2008). SMCHD1 was first identified in a mouse mutagenesis screen for modifiers of the variegated expression of a multi-copy transgene (Bleweitt et al., 2005). Gene targeting confirmed that SmcHD1 was necessary for hypermethylation of CpG islands associated with X-inactivation, and continued association of the SmcHD1 protein with the inactive X suggested its continuous requirement in maintaining X inactivation (Blewitt et al., 2008). The inventors' observations paint a strikingly similar picture for SMCHD1 and the D4Z4 arrays: SMCHD1 is necessary for D4Z4 hypermethylation, SMCHD1 remains associated with the D4Z4 array in skeletal muscle cells, and its continuous expression is required to maintain array silencing. It will be interesting to examine individuals with SMCHD1 mutations for subclinical abnormalities of X-inactivation.

The SmcHD1 mutation was originally called the Momme D1 (Modifiers of Murine Metastable Epialleles D1) locus (Blewitt et al., 2005). The term metastable epiallele has been applied to genes that show variable expression because of probabilistic determinants of epigenetic repression (Rakyan et al., 2002). An example of a metastable epiallele in mice is the agouti viable yellow (A^{vy}) locus; coat colors of isogenic mice can vary based on the epigenetic state of a retrotransposon integrated near the agouti promoter (Duhl et al., 1994). SmcHD1 is a modifier of metastable epialleles because SmcHD1 haploinsufficiency increased the penetrance of agouti expression (Blewitt et al., 2005). In the case of FSHD, decreased levels of SMCHD1 resulted in decreased D4Z4 CpG methylation and variegated expression of DUX4 in myonuclei. In both FSHD1 and FSHD2, the penetrance is incomplete (the inventors identified five asymptomatic carriers of an SMCHD1 variant and a permissive D4Z4 haplotype (Table 3), and the presentation is often asymmetric. Both features are consistent with FSHD as a metastable epiallele disease. The demonstration that independently variable modifiers of D4Z4 chromatin relaxation (repeat size for FSHD1 and SMCHD1 activity for FSHD2) regulate the penetrance of variegated expression of DUX4, suggests that DUX4 should be regarded as a metastable epiallele causing phenotypic variation in humans.

TABLE 3
Non penetrant carriers

| Family | Individual | Gender | age | FseI | Units |
|---|---|---|---|---|---|
| Rf392 | 102 | F | 54 | 17 | 50 U |
| Rf393 | 101 | M | 75 | 11 | 89 U |
| Rf393 | 206 | F | 42 | 11 | 18 U |
| Rf393 | 302 | F | 27 | 19 | 18 U |
| Rf393 | 303 | M | 25 | 19 | 18 U |
| Rf393 | 305 | M | 34 | 21 | 20 U |

Information on Non-Penetrant SMCHD1 Variant Carriers with a Permissive D4Z4 Haplotype.

Indicated are family ID, individual ID, gender, age, FseI methylation level and D4Z4 array size in units (U) of smallest permissive allele.

The disease mechanisms of FSHD1 and FSHD2 converge at the level of D4Z4 chromatin relaxation and the variegated expression of DUX4 (van der Maarel et al., 2011; Geng et al., 2012). Both FSHD1 and FSHD2 require inheritance of two independent genetic variations: a version of the DUX4 gene with a polyadenylation signal and a second genetic variant that results in D4Z4 chromatin relaxation. For FSHD1 the genetic variant associated with chromatin relaxation is contraction of the D4Z4 array and is therefore transmitted as a dominant trait. For FSHD2, genetic variants of SMCHD1, which is on chromosome 18, segregate independently of the FSHD-permissive DUX4 allele on chromosome 4 and result in a digenic inheritance pattern in affected kindreds. Considering the variable penetrance and asymmetric disease presentation, as well as the FSHD2 families without SMCHD1 variants, it is likely that other modifier loci will be identified and that SMCHD1 mutations could also modify the penetrance of FSHD1. Moreover, many other human diseases show variable penetrance that might be related to epigenetic control. SMCHD1 variants may modify the epigenetic repression of other genomic regions and the penetrance of other human diseases as well.

### Materials and Methods

Methods Summary.

D4Z4 CpG methylation was analyzed as described previously (Lemmers et al., 2010) with modifications as described in detail below. Targeted capture and massive parallel sequencing was performed on DNA extracted from peripheral blood that was used for construction of a shotgun sequencing library. Enriched libraries were then sequenced on an Illumina Genome Analyzer II to get either single-end or paired-end reads. Variants were confirmed by Sanger sequencing of PCR-amplified DNA and cDNA from RNA isolated from primary myoblast cultures from FSHD2 patients and controls. Western blots were generated from protein lysates of primary fibroblast cultures and incubated with commercially available primary and secondary antibodies. RNA interference of SMCHD1 was performed with commercially available shRNAs as described in detail below.

Development and Validation of D4Z4 Methylation Test for FSHD2.

Genomic DNA isolated from peripheral blood lymphocytes from a large panel of controls, sporadic patients with FSHD and FSHD families were included in this study after obtaining informed consent. The clinical diagnosis of FSHD was based on a standardized clinical form made available through the Fields Center on world wide web at urmc.rochester.edu/fields-center/). For all individuals, the inventors performed a detailed genotyping, including D4Z4 repeat array length and chromosomal background analysis of chromosomes 4q and 10q.

The observation that in FSHD1 patients D4Z4 hypomethylation is restricted to the disease allele while in FSHD2 patients the repeats on all four chromosomes are affected provides a unique opportunity to develop a more sensitive and specific diagnostic test for FSHD2. Rather than separating the chromosome 4-derived fragments from the chromosome 10-derived fragments by using restriction enzyme Blnl, as done before (de Greef et al., 2009; van Overveld et al., 2003), a collective measurement of D4Z4 methylation on both chromosomes 4 and 10 should yield a more sensitive and specific diagnostic test for FSHD2. From previous tests involving three methylation-sensitive restriction enzymes, FseI was shown to be the most informative enzyme (de Greef et al., 2009; van Overveld et al., 2003). Therefore, the inventors redesigned the FseI D4Z4 methylation test so that it interrogates all four alleles simultaneously by omitting BlnI from the digestion (FIG. 4). It has been previously shown that the FseI methylation value of the first D4Z4 unit in controls is ˜50% on both chromosomes 4q (de Greef et al., 2009; van Overveld et al., 2003). The average FseI methylation level of the first unit in pathogenic chromosomes 4 in FSHD1 patients (n=21) was shown to be 20% (van Overveld et al., 2005) while in FSHD2 patients the inventors found for both chromosomes 4 on average a value of 13% (n=32) (de Greef et al., 2010). While in controls and FSHD1 patients, the inventors would expect (near-) normal methylation values (as in FSHD1 the hypomethylation signal from the disease allele would be diluted 3× by the normal methylation levels of the normal chromosome 4 and chromosomes 10), in FSHD2 patients, the inventors would expect to see profound hypomethylation. As the activity of restriction enzymes is sensitive to salt or protein impurities in the gDNA the inventors introduced an extra DNA clean-up step preceding digestion with FseI. This extraction column-based purification step can also be applied to gDNA embedded in agarose plugs and to samples with low gDNA concentrations.

Upon digesting with BglII a 4061 bp fragment is released (M in FIG. 4c) while digesting with FseI yields a fragment of 3387 bp when the restriction site is unmethylated (U in FIG. 4c). The previously used enzyme BlnI to separate chromosomes 4 (white) from chromosomes 10 (black) is also shown.

To validate the modified methylation test, the inventors re-analyzed the same gDNA samples from a previous study (de Greef et al., 2010). While the inventors obtained nearly identical average methylation levels in all three populations analyzed, the modified methylation test clearly improves discrimination between FSHD1 and FSHD2 by reducing the error bars particularly in FSHD1 patients (FIG. 4d). FIG. 4b shows a typical example of the D4Z4 methylation analysis on a de novo FSHD2 patient and his unaffected family members. The FSHD2 patient has comparable methylation levels (%) to her unaffected mother who carries a non-permissive alleles (NP) only. The unaffected father has significant lower methylation levels than mother and daughter as quantified bp fragment intensities.

To define threshold values for D4Z4 methylation the results shown in FIG. 4d were expanded to 72 controls, 93 FSHD1 patients and 53 FSHD2 patients. As shown in FIG. 4e, the average methylation value is 44% for control individuals and 33% for patients with FSHD1. FSHD2 patients show an average D4Z4 methylation value of 11.6% with a standard deviation (SD) of 4.7%. The threshold value for FSHD2 was defined as 24.3%, being 2SD below the average of the general control population.

Cases and Samples.

Forty-one FSHD2 patients were selected based on published clinical and molecular criteria (de Greef et al., 2009; (de Greef et al., 2010) and D4Z4 methylation levels <25% as described in the previous section (Table 1). Assessment of the FSHD2 phenotype was determined by experienced neurologists (RT, BGME, GWP, SS, CD, MV). Initial testing was performed at Leiden University Medical Center using Pulsed Field Gel electrophoresis and hybridization of Southern blots with P13E-11, “A” and “B” probes, and SSLP length determined using an ABI Prism 3100 Genetic analyzer (Lemmers et al., 2007; Lemmers et al., 2001; Lemmers et al., 2010). Forty of them had D4Z4 array sizes >10 units on both chromosomes 4 ruling out FSHD1. One patient had 2 contracted alleles on chromosome 10 possibly explaining the low D4Z4 methylation and was therefore excluded from further studies. Of the 39 remaining families, of 13 the inventors had sufficient family information suggesting dominant inheritance of the D4Z4 hypomethylation and in 7 cases the hypomethylation appeared to have occurred de novo (see FIG. 5 for pedigrees). For exome sequencing, the inventors selected 5 families with a dominant inheritance pattern and 2 with de novo hypomethylation in the patient. In total 14 individuals from these families were analyzed by exome sequencing (indicated with grey boxes in FIG. 5). All participants provided written consent, and the Institutional Review Boards of participating institutes approved all studies.

D4Z4 Methylation Analysis.

D4Z4 arrays were analyzed for their methylation state using the methylation sensitive endonuclease FseI largely as previously described but with omission of BlnI and inclusion of an extra DNA purification step (van Overveld et al., 2003) (see also previous section: development and validation of D4Z4 methylation test for FSHD2). Briefly, genomic DNA was prepared from peripheral blood lymphocytes using standard protocols. The DNA was double digested with EcoRI and BglII overnight at 37° C. and cleaved DNA was bound to DNA purification columns (according to the manufacturer instructions), washed, and eluted for subsequent 4 hour digestion with FseI. EcoRI/BglI/FseI digested DNA fragments were separated by size on 0.8% agarose gels, transferred to a nylon membrane (Hybond XL, Amersham) by Southern blotting and probed using the p13E-11 radiolabeled probe (Wijmenga et al., 1992). Probe signals were quantified using a phosphorimager and Image Quant software. The signal from the total amount of hybridizing EcoRI digested fragments (4061 bp and 3387 bp fragment) was divided by the signal quantity from the 4061 bp fragment to give the percentage of P13E-11 hybridizing fragments that contain methylated FseI sites.

Exome Definition, Array Design and Target Masking.

The inventors targeted all protein-coding regions as defined by RefSeq 36.3. Entries were filtered for the following: (i) CDS as the feature type, (ii) transcript name starting with “NM_” or “−”, (iii) reference as the group_label, (iv) not being on an unplaced contig (for example, 17|NT_{—}113931.1). Overlapping coordinates were collapsed for a total of 31,922,798 bases over 186,040 discontiguous regions. A single custom array (Agilent, 1M features, aCGH format) was designed to have probes over these coordinates as previously described, except here, the maximum melting temperature (Tₘ) was raised to 73° C.

The mappable exome was also determined as previously described using this RefSeq exome definition instead. After masking for ‘unmappable’ regions, 30,923,460 bases were left as the mappable target.

Targeted Capture and Massive Parallel Sequencing.

Genomic DNA was extracted from peripheral blood lymphocytes using standard protocols. Five micrograms of DNA from each of the eight individuals was used for construction of a shotgun sequencing library as described previously using paired-end adaptors for sequencing on an Illumina Genome Analyzer II (GAII). Each shotgun library was hybridized to an array for target enrichment; this was then followed by washing, elution and additional amplification. Enriched libraries were then sequenced on a GAII to get either single-end or paired-end reads.

Read Mapping and Variant Analysis.

Reads were mapped and processed largely as previously described. In brief, reads were quality recalibrated using Eland and then aligned to the reference human genome (hg19) using Maq. When reads with the same start site and orientation were filtered, paired-end reads were treated like separate single-end reads; this method is overly conservative and hence the actual coverage of the exomes is higher than reported here. Sequence calls were performed using Maq and these calls were filtered to coordinates with ≧8× coverage and consensus quality ≧20.

Indels affecting coding sequences were identified as previously described, but the inventors used phaster instead of cross_match and Maq. Specifically, unmapped reads from Maq were aligned to the reference sequence using phaster (version 1.100122a) with the parameters −max_ins:21 −max_del:21 −gapextend_ins:−1 −gapextend_del:−1 −match_report_type:1. Reads were then filtered for those with at most two substitutions and one indel. Reads that mapped to the negative strand were reverse complemented and, together with the other filtered reads, were remapped using the same parameters to reduce ambiguity in the called indel positions. These reads were then filtered for (i) having a single indel more than 3 bp from the ends and (ii) having no other substitutions in the read. Putative indels were then called per individual if they were supported by at least two filtered reads that started from different positions. An ‘indel reference’ was generated as previously described, and all the reads from each individual were mapped back to this reference using phaster with default settings and −match_report_type:1. Indel genotypes were called as previously described.

To determine the novelty of the variants, sequence calls were compared against 1200 individuals for whom the inventors had previously reported exome data, and to the 1000 genomes database dbSNP. Annotations of variants were based on NCBI and UCSC databases using an in-house server (SeattleSeqAnnotation). Loss-of-function variants were defined as nonsense mutations (premature stop) or frame-shifting indels. For each variant, the inventors also generated constraint scores as implemented in GERP.

Post Hoc Ranking of Candidate Genes.

Candidate genes were ranked by summation of variant scores calculated by counting the total number of nonsense and nonsynonymous variants across the five FSHD2 exomes.

Mutation Validation.

Sanger sequencing of PCR amplicons (LGCT, Leiden, Netherlands) from genomic DNA was used to confirm the presence and identity of variants in the candidate gene identified via exome sequencing and to screen the candidate gene in affected and unaffected family members of FSHD2 families.

Cells and Culture Conditions.

Primary human myoblasts were obtained through the Fields Center at the University of Rochester (available on the world wide web at urmc.rochester.edu/fields-center/protocols/myoblast-cell-cultures.cfm). Biopsies were obtained after full consent with an IRB-approved protocol. Consents included the possibility of exome sequencing and sharing of samples with other investigators. Normal human myoblasts were grown on dishes coated with 0.01% Calf skin collagen (Sigma Aldrich, St. Louis, Mich.) in F10 medium (Invitrogen) supplemented with 20% FBS, 100 U/ml penicillin and 100 μg/ml streptomycin, 4 μg/ml bFGF (Invitrogen), and 1 μM dexamethasone (Sigma Aldrich) (Snider et al., 2010), in a humidified atmosphere containing 5% CO2 at 37° C. Transduction of human myoblasts with retroviral vectors was accomplished by seeding cells at 5×10⁴ cells/cm² density on day −1. On Day 0 the medium is changed and cells are incubated with vector preparations and polybrene (4 μg/ml, Sigma Aldrich). 2-4 hours later the medium is replaced with a fresh sample and cells are cultured and split at ˜75% confluence to prevent differentiation. Human myoblasts transduced with pGIPZ shRNA expression vectors were selected with puromycin (0.5 μg/ml). Differentiation was induced using F10 medium supplemented with 1% horse serum and ITS supplement (insulin 0.1%, 0.000067% sodium selenite, 0.055% transferrin; Invitrogen).

Fibroblast obtained from FSHD2 patients and family members were cultured in DMEM/F-12 media supplemented with 20% heat inactivated fetal bovine serum, 1% penicillin/streptomycin, 10 mM HEPES, 1 mM Sodium Pyruvate (all Invitrogen).

RNA Extraction and cDNA Synthesis.

Total RNA was extracted using the Qiagen miRNeasy mini isolation kit with Dnasel treatment. The RNA concentration was determined on a ND-1000 spectrophotometer (Thermo Scientific, Wilmington, USA) and the quality was analyzed with a RNA 6000 Nanochip Labchip on an Agilent 2100 BioAnalyzer (Agilent Technologies Netherlands BV, Amstelveen, The Netherlands). cDNA was synthesized from 2 μg of total RNA using random hexamer primers (Fermentas, St Leon-Rot, Germany) and the RevertAid H Minus M-MuLV First Strand Kit (Fermentas Life Sciences, Burlington, ON, Canada) according to the manufacturer's instructions. After the cDNA reaction 30 μL of water was added to an end volume of 50 μL. All primers used for SMCHD1 mutation analysis and real-time PCR were designed using Primer 3 software. Primer sequences are in Table 4.

TABLE 4

| Name | Sequence | position | SEQ ID NO: |
|---|---|---|---|
| exon 8F | ATTTCCACCTTTGGACACGA | NM015295_1202F | 10 |
| exon 12R | TGCTGACCTGGAATTTGTCA | NM015295_1756R | 11 |
| exon 14F | TCCCCTCTTTTATGGAAGCAT | 2052F | 12 |
| exon 17R | TTCCTGGAAGCTTTTGCATT | 2395R | 13 |
| exon 18F | CATGGAGGAAAATGGCCTTA | NM015295_2481R | 14 |
| exon 20F | ATTCAGCCAGTTCTTGAAGC | NM015295_2770F | 15 |
| exon 21/22R | TGCCTTGACAAGAGTTTACAGG | NM015295_2886R | 16 |
| exon 24F | TCTGGAACCAGTATTTTAACAGGA | (3151F) | 17 |
| exon 25F | GGATAGCGGGTGATATTATGC | (3368F) | 18 |
| exon 26R | TTGCACATCAGGAAGCAGAC | (3518R) | 19 |
| exon 28F | CTGGGGTTGGACTTGATAGC | (3779F) | 20 |
| exon 28R | AACCCCAGCAATTGACAAAG | NM015295_3792R | 21 |
| exon 30R | GGTGCTGGATTATCCCACTG | (4070R) | 22 |
| exon 31R | CTGTTGGTTTGAAGGCATGA | NM015295_4137R | 23 |
| exon 35F | TCCAGTTTGGTTTTATGATGGA | (4574F) | 24 |
| exon 37R | TTCACGAAGGGGAATTCAAG | (4889R) | 25 |
| exon 39R | TAAGTGCTGCCATTTGTTGC | NM015295_5068R | 26 |
| exon 47F | CGACAGATTGTCCAGTTCCTC | (6125F) | 27 |
| exon 48R | CCAATGGCCTCTTCTCTCTG | (6225R) | 28 |
| exon 12F | TCCTAAGAAGAGAGGGCTTGC | NM015295_1671F | 29 |
| exon | TTAATCTCAGGAGTCCAATTAACTTT | (3488R) | 30 |
| 25/26WTR | | | |
| exon 29F | TCCAGGTCCTCCTGGAAATA | (3864F) | 31 |
| exon 30F | TCCAGCACCGGTACAACAT | NM015295_4062F | 32 |
| exon 36F | CCTGCCTAATCAACCTGTGAA | (4638F) | 33 |
| exon 36R | TACTGGCAACTGAGCGAACA | (4720R) | 34 |
| exon 43F | CCCATTGGAGATCCAGTCTT | NM015295_5596F | 35 |
| exon 44R | CCGCATCCAGATTATCCAAA | NM015295_5716R | 36 |
| exon 45F | TGGATAAACTTCGGGGAATG | (5837F) | 37 |

Quantification of mRNA Levels Using Real-Time RT-PCR.

The mRNA levels were measured in duplo by real-time PCR using a SYBR Green QPCR master mix kit (Stratagene) on a MyiQ (Biorad Laboratories, Veenendaal, The Netherlands) running an initial denaturation step at 95° C. for 3 min, followed by 40 cycles of 10 s at 95° C. and 45 s at 60° C. All PCR products were analyzed for specificity by melting curve analysis and on a 2% agarose gel. The results of the quantitative RT-PCR were analyzed and quantified using CFX optical system software version 2.0 (Biorad Laboratories, Veenendaal, The Netherlands). All expression levels were calculated using GAPDH and GUS primers as constitutively expressed standard for cDNA input, and the relative steady-state RNA levels of the genes of interest were calculated by the method of Pfaffl (2001). All primers were designed using Primer 3 software. Primer sequences are available upon request.

Semiquantitative RNA Analysis and Sequencing of SMCHD1 Variants.

Splicing alterations were analyzed by RT-PCR using different primer sets covering the exons surrounding the possible splice site variant. Subsequently, PCR fragments obtained from SMCHD1 variant carriers and control samples were analyzed on 1.5-2% agarose gels. Fragments were isolated from gel and analyzed by Sanger sequencing (LGTC).

Allelic expression analysis of missense variants (wild type versus variant allele) was done by Sanger sequencing (LGTC) by comparison of nucleotide peak heights of wild type and variant alleles.

DUX4 mRNA levels were analyzed in duplo by RT-PCR running an initial denaturation step at 95° C. for 6 min, followed by 35 cycles of 10 s at 95° C. and 30 s at 60° C. All PCR products were analyzed on a 2% agarose gel. All expression levels were corrected for GAPDH and GUS as constitutively expressed standard for cDNA input. All primers were designed using Primer 3 software. Primer sequences are available upon request.

Chromatin Immunoprecipitation.

Chromatin was prepared from myoblast cells lines fixed with 1% formaldehyde according to a published protocol (Nelson et al., 2006). Control and FSHD2 myoblast carried a comparable total number D4Z4 repeat units on permissive and nonpermissive chromosomes. 60 ug chromatin was incubated with the different antibodies. Every sample was independently studied twice. Antibodies against SMCHD1 (ab31865) and H3 (ab1791) were purchased from Abcam (Cambridge, Mass., USA). Normal rabbit serum was used to measure unspecific binding of proteins to beads. Immunopurified DNA was quantified with D4Z4 Q-PCR primer pair (Zeng et al., 2009) and quantitative PCR measurements were done with CFX96TM real time system using iQTM SYBRR Green Supermix. Relative enrichment values were calculated by subtracting the IgG ChIP values representing background from the ChIP values with the SMCHD1 and H3 antibody and SMCHD1 values were divided by H3 enrichment values for D4Z4 copy number correction.

Antisense-Mediated Exon Skipping.

Antisense oligonucleotides (AONs) for SMCHD1 exons 29 (29AON5 5′-GUC CAG AAA UUA GUU GCA CUC-3′ (SEQ ID NO:38)) and 36 (36AON1 5′-GAU UAG GCA GGA CUU CAA CU-3′ (SEQ ID NO:39)) were designed based on the guidelines for Duchenne Muscular Dystrophy (DMD) exons (Aartsma-Rus, 2012). All AONs target exon-internal sequences and consist of 2′-O-methyl RNA with a full-length phosphorothioate backbone and were manufactured by Eurogentec (Seraing, Belgium). Human control myoblasts were seeded in 6 wells plates or 6 cm dishes at a cell density of approximately 1*10⁴ cells per cm² and cultured for 2 days. Myotubes were obtained by growing 70% confluence myoblasts for 4 days on differentiation media (DMEM (+glucose, +L-glutamin, +pyruvate), 2% horse serum). Four hours after the differentiation medium was added AONs were transfected at a 250 nM concentration, using 2.5 μl polyethyleneimine (MBI-Fermentas, Leon-Rot, Germany) per μg AON according to the manufacturer's instructions. A FAM-labeled AON targeting exon 50 of the DMD gene (h50AON2 5′-GGC UGC UUU GCC CUC-3′ (SEQ ID NO:40)) was used to confirm the efficiency of transfection and exon skipping. Primers flanking the targeted exons were used to study splicing of the SMCHD1 or DMD gene.

Knock Down of SMCHD1 mRNA's in Normal Human Myoblasts.

SMCHD1 transcripts were targeted for degradation using lentiviral vectors expressing short hairpin RNA's from a CMV promoter and linked to a puromycin selection cassette by an internal ribosome entry site (IRES). Five different pGIPZ (Open Biosystems, Huntsville, Ala.) vectors were purchased and each was tested in normal human myoblasts for the effect on SMCHD1 transcripts by quantitative PCR, immunofluorescence signal intensity, and western blot.

Antibodies, Immunofluorescence and Western Blotting.

Immunofluorescence for human DUX4 was performed using a rabbit monoclonal C-terminal specific antibody (Epitomics E5-5) as previously described (Geng et al., 2012). Immunoreactivity was detected with a mouse anti-rabbit secondary antibody conjugated to Alexa Fluor 594 (Molecular Probes, 1:1000 dilution).

For western blotting, fibroblast or myoblast lysates were run on a 7.5% SDS-PAGE and transferred to PVDF membrane. SMCHD1 protein was detected using a commercially available rabbit polyclonal antibody (Sigma, HPA039441 (1:250 dilution)), and as reference protein tubulin was detected with a commercially available mouse monoclonal antibody (Sigma, T6199 (1:2000)). Bound antibodies were detected with an HRP-conjugated donkey anti-rabbit (Pierce, 31458 (1:5000)) and an IRDye 800CW-conjugated goat anti-mouse antibody (Westburg, 926-32210 (1:5000)), respectively.

### Example 2

The inventors investigated whether SMCHD1 may act as a modifier for disease severity in FSHD1 families and may have a role in the marked variability of clinical expression that is encountered in some families.

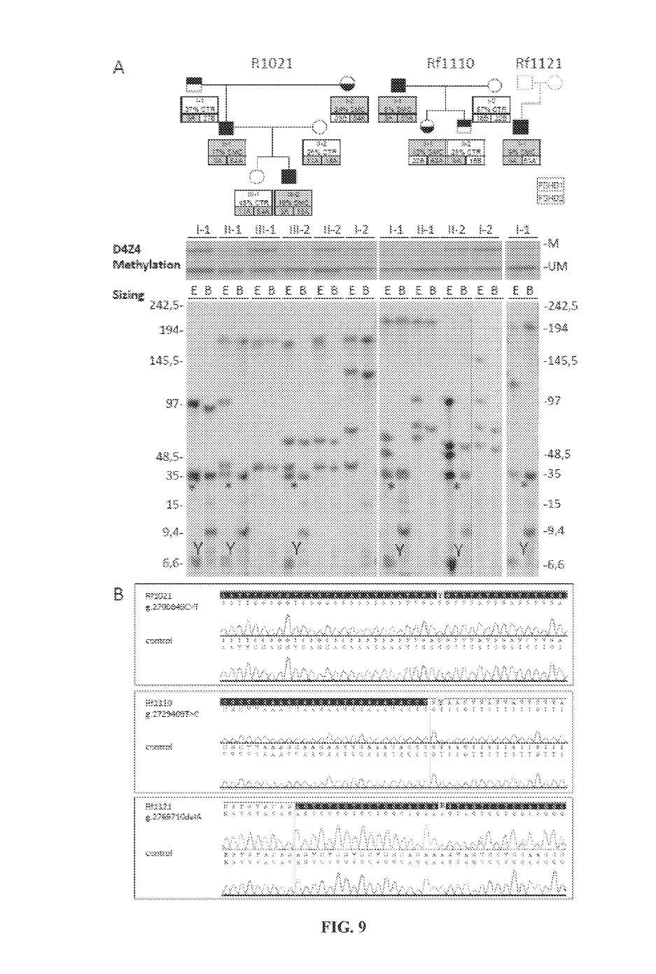

To identify modifiers of disease severity, of particular interest are those FSHD1 families carrying upper-sized D4Z4 repeat arrays of 8-10 units, since carriers of these alleles are more likely to have a partial or less severe form of FSHD, or to be asymptomatic.40-42 To explore the possibility that mutations in SMCHD1 may modify the disease severity in FSHD1 families, the inventors investigated the SMCHD1 locus in 3 independent FSHD1 patients with a repeat array of 9 D4Z4 units on a FSHD permissive DUX4 PAS containing chromosome and an unusually severe clinical presentation of the disease. The relevant biometric and genetic observations in these families are summarized in Tables 5 and 6. Pedigrees of these three families are presented in FIG. 9A.

TABLE 5
Genetic and epigenetic data from the three families

| | | SMCHD1 | SMCHD1 | SMCHD1 | | | Fsel | | | | |
|---|---|---|---|---|---|---|---|---|---|---|---|
| Rf | Nr | gene (GRCh37) | cDNA | protein | Position | Type | % | 4q-1 | 4q-2 | 10q-1 | 10q-2 |
| Rf1021 | I-1 | None | — | — | — | — | 37 | 9U | 27U | 9U | 27U |
| | | | | | | | | 4A161 | 4B168 | 10A166 | 10A166 |
| | I-2 | g.2700849C>T | c.1580C>T | p.Thr527Met | Exon 12 | Missense | 24 | 39U | 54U | 11U | 21U |
| | | | | | | | | 4Q163 | 4A161 | 10A166 | 10A166 |
| | II-1 | g.2700849C>T | c.1580C>T | p.Thr527Met | Exon 12 | Missense | 17 | 9U | 54U | 11U | 27U |
| | | | | | | | | 4A161 | 4A161 | 10A165 | 10A166 |
| | II-2 | None | — | — | — | — | 26 | 11U | 18U | 53U | 54U |
| | | | | | | | | 4A161 | 4A161 | 10A166 | 10A166 |
| | III-1 | None | — | — | — | — | 45 | 11U | 54U | 11U | 54U |
| | | | | | | | | 4A161 | 4A161 | 10A166 | 10A166 |
| | III-2 | g.2700849C>T | c.1580C>T | p.Thr527Met | Exon 12 | Missense | 10 | 9U | 18U | 11U | 53U |
| | | | | | | | | 4A161 | 4A161 | 10A166 | 10A166 |
| Rf1110 | I-1 | g.2729409T>C | c.3048+2T>C | | Intron24 | Splice | 8 | 9U | 63U | 15U | 19U |
| | | | | | | site | | 4A161 | 4A161 | 10A166 | 10A166 |
| | I-2 | None | — | — | — | — | 57 | 16U | 22U | 30U | 43U |
| | | | | | | | | 4B163 | 4B168 | 10A166 | 10A166 |
| | II-1 | g.2729409T>C | c.3048+2T>C | | Intron24 | Splice | 10 | 22U | 63U | 19U | 30U |
| | | | | | | site | | 4B168 | 4A161 | 10A166 | 10A166 |
| | II-2 | None | — | — | — | — | 28 | 9U | 16U | 15U | 30U |
| | | | | | | | | 4A161 | 4B163 | 10A166 | 10A166 |
| Rf1121 | I-1 | g.2769710delA | c.4738del | p.Ser1580fs | Exon 38 | Deletion | 9 | 9U | 59U | 34U | 34U |
| | | | | | | | | 4A161 | 4A166 | 10A164 | 10A166 |

TABLE 6
Clinical features

| | | | | Age at | | MMT | |
|---|---|---|---|---|---|---|---|
| Rf | Nr | Sex | AAE | onset | CSS | score | Diagnosis |
| Rf1021 | I-1 | M | 71 | Unknown* | 3 | 261/300 | FSHD1 |
| | I-2 | F | 67 | Unknown* | 4 | 255/300 | FSHD2 |
| | II-1 | M | 48 | 12 | 10 | 65/300 | FSHD1 + |
| | | | | | | | FSHD2 |
| | II-2 | F | 38 | — | 0 | 300/300 | Unaf- |
| | | | | | | | fected |
| | III-1 | F | 15 | — | 0 | 300/300 | Unaf- |
| | | | | | | | fected |
| | III-2 | M | 6 | 5 | 6 | 198/300 | FSHD1 + |
| | | | | | | | FSHD2 |
| Rf1110 | I-1 | M | 55 | 6 | 10 | 55/300 | FSHD1 + |
| | | | | | | | FSHD2 |
| | I-2 | F | 53 | — | 0 | 300/300 | Unaf- |
| | | | | | | | fected |
| | II-1 | F | 26 | 24 | 5 | 280/300 | FSHD2 |
| | II-2 | M | 21 | Unknown* | 2 | 269/300 | FSHD1 |
| Rf1121 | II-1 | M | 67 | 15 | 10 | 75/300 | FSHD1 + |
| | | | | | | | FSHD2 |

CSS: Clinical Severity Score,
AAE: age at the last examination;
MMT: Manual Muscle Testing
*These patients do not report any symptom

In the first family, Rf1021, the proband (II-1) has been followed since the age of 35 when he became wheelchair-dependent. He started to experience asymmetric scapular weakness at the age of 18, but he reported inability to blow in a flute at the age of 12. During the last examination, at age 48, he presented with severe and asymmetric facial weakness of orbicularis oculi and oris, marked shoulder girdle weakness associated with bilateral scapular winging and humeral weakness. He displays marked hyperlordosis due to abdominal muscle weakness and weakness and atrophy of lower legs. Distal upper limb muscles are also becoming involved. He has a clinical severity score (CSS) of 10.11 Genetic analysis showed that this proband carries a 9 D4Z4 unit 4A161 allele confirming the diagnosis of FSHD1. His 6 year old son (III-2) was referred because of difficulties in raising his arms and a history of frequent falls. At examination he presented with weakness of the orbicularis oculi muscles, mild shoulder girdle weakness with scapular winging, Gower's sign and asymmetric foot dorsiflexor weakness (CSS 6).

The inventors also examined the older (III-1) daughter, 15 years old, and concluded that she was clinically unaffected. The father of the proband (1-1), at age 71, displayed mild facial and asymmetric shoulder girdle weakness (CSS 3) and mother of the proband (1-2: 67 years old) showed asymmetric facial weakness and shoulder girdle involvement (CSS 4). Neither of them was complaining of any symptom except for arm fatigability. Extensive genotype, methylation and SMCHD1 mutation analysis in this family showed that the proband's father is a mildly affected FSHD1 patient carrying the 9 D4Z4 units of a 4A161 allele without a mutation in the SMCHD1 gene, consistent with the intermediate D4Z4 methylation levels. The proband's mother, also mildly affected, is an FSHD2 patient carrying a normal-sized 4A161 allele and a mutation in the SMCHD1 gene (c.1580C>T). This mutation has not been reported previously in dbSNP or the 1000 Genomes Project and most probably results in a change of the amino acid Threonine to Methionine at protein position 527. The proband and his affected son have inherited alleles for both FSHD1 (9 units 4A161 allele) and FSHD2 (marked hypomethylation of D4Z4 loci associated with c.1580C>T mutation in SMCHD1), suggesting a possible explanation for the severity of clinical phenotype in the proband and the unusual early onset in the son. In the second family, Rf1110, the proband (I-1) was examined at the age of 56 showing, since the age of 6, a progressive and asymmetric weakness of facial, shoulder girdle muscle weakness associated with scapular winging and humeral muscle weakness. Abdominal weakness was also observed with marked hyperlordosis and pelvic girdle muscle weakness.

Upon examination, the lower legs muscles were also affected (CSS 10). The patient became wheelchair-dependent by the age of 39. The diagnosis of FSHD1 was confirmed by the presence of a D4Z4 repeat array of 9 units on a 4A161 allele. His mother and father have no history of muscle disease and have not been examined. His son (II-2), 21 years old, was diagnosed as an asymptomatic FSHD1 carrier since he inherited the contracted 4A161 allele. At examination he showed very mild facial weakness and shoulder girdle involvement with mild right scapular winging (CSS 2). The older daughter (II-1), 26 years old, did not inherit the contracted D4Z4 repeat array of 9 units and was considered unaffected. On clinical examination, however, she had mild asymmetric facial weakness, scapular winging and right anterior leg weakness. Functionally, this patient's only complaints are fatigability in right foot dorsiflexion and in arm raising above her head (CSS 5). Methylation studies revealed slightly lower D4Z4 methylation levels in 11-2, consistent with FSHD1 diagnosis and a marked hypomethylation in patients I-1 and II-1, suggestive of FSHD2. These results prompted us to perform SMCHD1 mutation analysis resulting in the identification of a mutation in both individuals in a highly conserved nucleotide of the 5′ splice consensus site of exon 24, which has not been reported in any of the public databases. In summary, in this family, the proband (1-1), carries the diagnosis of FSHD1 and FSHD2 and has a severe phenotype. The proband's daughter (II-1), diagnosed with FSHD2, and his son (II-2), diagnosed with FSHD1, are only mildly affected.

In the last family, Rf1121, only individual II-1 was available for examination. He has no family history of muscle disease and no offspring. At age 15 he was noted to have asymmetric facial weakness of orbicularis oculi and oris muscles, shoulder girdle involvement with scapularwinging, and abdominal muscle weakness. Anterior lower legs weakness appeared at the age of 30 and subsequently pelvic girdle muscles were involved. At the age of 33 he was walking with a cane. At the age of 40 he became wheelchair-dependent and was unable to lift his arms. At his most recent examination, at age 65, his CSS was 10. Extensive genotyping of D4Z4 repeats on chromosomes 4 and 10 showed 9 D4Z4 units on a 4A161 allele confirming the diagnosis of FSHD1. Methylation studies revealed profound D4Z4 hypomethylation and SMCHD1 mutation analysis identified a single nucleotide deletion in exon 38 which is predicted to result in a frameshift in the SMCHD1 open reading frame confirming the additional diagnosis of FSHD2. In conclusion, these genetic and clinical data suggest that a mutation in SMCHD1 gene can act as a modifier of disease severity in FSHD 1 patients.

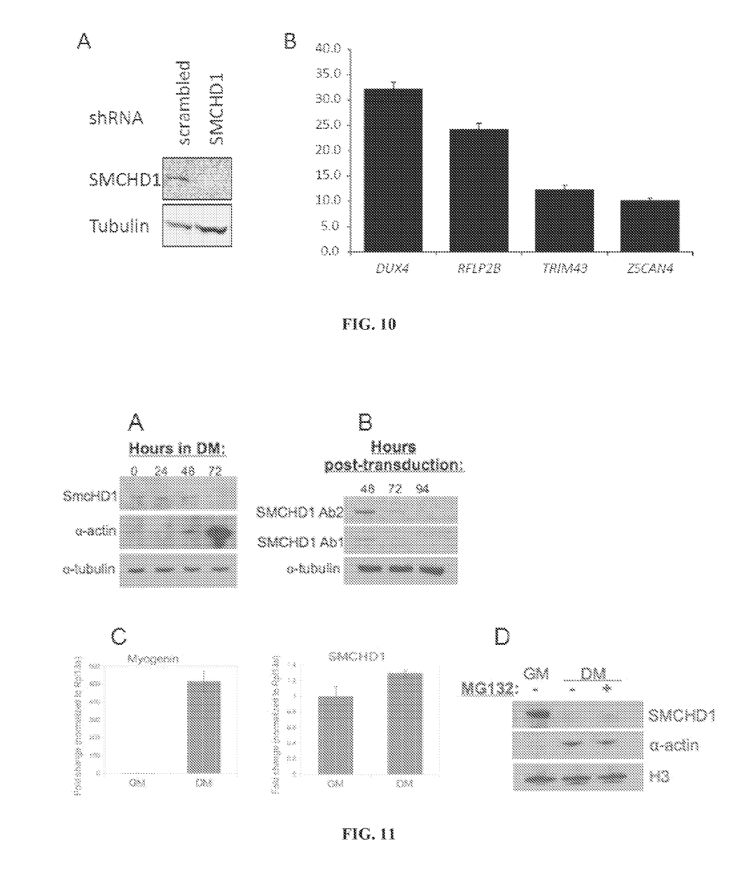

Previously, the inventors showed that depletion of SMCHD1 by RNA interference in control myotubes carrying a normal sized D4Z4 repeat leads to the transcriptional activation of DUX4.33 According to clinical observations implying a synergistic effect of a D4Z4 contraction and a SMCHD1 mutation on transcriptional derepression of DUX4, the inventors hypothesized that depletion of SMCHD1 in FSHD1 myotubes may result in transcriptional activation of DUX4 beyond that observed in FSHD1 myotubes, potentially resulting in a more severe phenotype. The inventors tested this hypothesis by lentiviral transduction of SMCHD1 shRNAs into an FSHD1 myotube and observed increased levels of DUX4 mRNA in myotubes with sufficient SMCHD1 knockdown (FIG. 10).

The observations in the present study, in addition to suggesting that SMCHD1 can act as a disease modifier, further support the hypothesis that FSHD1 and FSHD2 share a common pathophysiologic pathway. Individuals with combined FSHD1 and FSHD2 genetic defects have a more severe clinical phenotype than expected based on the borderline repeat size of the FSHD1 allele whereas patients within the same family with only the FSHD1 borderline allele or the FSHD2 genetic defect were less severely affected. These findings support an additive effect of the SMCHD1 mutation on the pathophysiological pathway triggered by D4Z4 repeat array contraction.

### Materials And Methods

Families

The local ethical committees of the involved institutes approved this study. All patients and their relatives signed an informed consent. The families described in this study were identified from a cohort of 42 unrelated FSHD1 patients. Of these, 3 have D4Z4 methylation levels <25% indicative for FSHD2. Three of them have a mutation in SMCHD1 and are described in this study. Clinical evaluation included Manual Muscle Testing score of 60 muscles and Clinical Severity Score. Exclusion of mutations in CAPN3, VCP and FHL1 was done by direct sequencing. All other muscular dystrophies and myopathies that may resemble FSHD were excluded by western blotting as described previously.¹

D4Z4 Analysis.

An comprehensive genotype of the D4Z4 region on chromosomes 4 and 10 was obtained by analysis of the size of the repeat arrays by pulsed field gel electrophoresis analysis, single sequence length polymorphism (SSLP) analysis at the proximal end of the repeat arrays and determination of the distal variation A or B, as previously described₂ (see also the Fields Center for FSHD Research Website available on the world wide web at urmc.rochester.edu/fields-center/ for detailed protocols). The repeat lengths of the FSHD1-sized D4Z4 repeat arrays was confirmed using Southern blot analysis of genomic DNA digested with EcoRI and double digested with EcoRI and BlnI separated by conventional linear gel electrophoresis, using a 5 kb ladder as DNA size standard (Biorad, 170-3624). Methylation levels in the proximal D4Z4 repeat units of chromosomes 4q and 10q was performed as reported.3 Mutation analysis of SMCHD1 gene was performed as described in a previous study.₄

SMCHD1 Mutation Analysis

Sanger sequencing of PCR amplicons (LGTC, Leiden, Netherlands) from genomic DNA was used to identify mutations in SMCHD1 and to screen the mutation in affected and unaffected family members of FSHD2 families. Exonic amplicons were amplified using intronic M13-tailed PCR primers. Primer sequences are available on request.

RNA Analysis

Total RNA extraction and cDNA synthesis were performed as described previously.⁴ For cDNA synthesis, 2 μg of total RNA was used and after the reaction 30 μL of water was added to an end volume of 50 pt. The mutations in the different families were studies using primers 5′-GAA TGT TTT TGG AAT GGA CGA-3′ and 5′-TCC ATC ATG ATC GCC ATA AA-3′ (Rf1021); 5′-GGA ACA GCT TTC CCA TTT CA-3′ and 5′-TTG CAC ATC AGG AAG CAG AC-3′ (Rf1110) and 5′-TCC AGT TTG GTT TTA TGA TGG A-3′ and 5′-TAA GTG CTG CCA TTT GTT GC-3′ (Rf1121).

SMCHD1 Depletion in FSHD1 Myoblasts.

Three unrelated FSHD1 myoblasts, were obtained from the Fields Center and grown as previously described.⁴ Detailed analysis revealed an FSHD-permissive DUX4-PAS containing chromosome with a repeat size of 6 units (FSHD1a and FSHD1b), or 4 units (FSHD1c). Two patients (FSHD1b and FSHD1c) carry a 4qB haplotype on their homologous chromosome and therefore DUX4 expression is only possible from their FSHD1 allele (see Table 7).² FSHD1 myoblasts carrying a FSHD-permissive DUX4-PAS containing chromosome with a normal sized repeat were obtained from the Fields Center and grown as previously described.4 Myoblast cultures were transduced with lentiviral particles harbouring scrambled or SMCHD1 specific shRNA constructs 48 hrs prior to differentiation as described previously.4 After differentiating into myotubes by serum starvation cells were harvested after 4 to 5 days and RNA and protein was extracted. cDNA was prepared and mRNA levels were measured by qRT-PCR as described. Reduction of SMCHD1 levels was confirmed by western blotting. Expression values were correlated to beta-glucuronidase.

TABLE 7

| | | Allele 4_1 | | Allele 4_2 | | Allele 10_1 | | Allele 10_2 | |
|---|---|---|---|---|---|---|---|---|---|
| sample | M/F | units | haplotype | units | haplotype | units | haplotype | units | haplotype |
| FSHD1_a | M | 6 | 4A161 | 11 | 4A168 | 16 | 10A166 | 23 | 10A176T |
| FSHD1_b | F | 6 | 4A161 | 52 | 4B168 | 18 | 10A166 | 21 | 10A166 |
| FSHD1_c | F | 4 | 4A161 | 13 | 4B163 | 9 | 10A166 | 19 | 10A166 |

### Example 3

### Post-Transcriptional Regulation Decreases the Abundance of SMCHD1 in Differentiating Muscle Cells

Preliminary studies indicate that SMCHD1 protein dramatically decreases during muscle cell differentiation (FIG. 11a) and this decrease can be reproduced in fibroblasts converted to skeletal muscle by the expression of MyoD (FIG. 11b), however, the SMCHD1 mRNA level does not decrease with differentiation (FIG. 11c). These findings are consistent with low levels of SMCHD1 immunodectection in skeletal muscle reported in the Human Protein Atlas (available on the world wide web at proteinatlas.org) and with our initial westerns showing very low levels of SMCHD1 in mouse muscle tissue compared to other tissues, such as brain (data not shown). Further preliminary studies suggest that the SMCHD1 mRNA is not efficiently translated in skeletal muscle cells: first, a pulse of 35-S-methionine shows significantly reduced incorporation into SMCHD1 in skeletal muscle cells compared to myoblasts (data not shown), indicating either decreased translation or rapid degradation of the translated product in skeletal muscle; and second, inhibiting protein degradation by the MG132 proteasome inhibitor did not result in stabilization of SMCHD1 protein in muscle cells (FIG. 11d). Although preliminary, these findings together support an inhibition of SMCHD1 mRNA translation in differentiation myotubes.

### Example 4

### Mouse Models for D4Z4 Chromatin Studies

To study whether the epigenetic regulation of D4Z4 is conserved, the inventors generated transgenic mice carrying normal sized D4Z4 arrays (12.5 units: D4Z4-12.5 mice) and FSHD-sized arrays (2.5 units: D4Z4-2.5 mice) with flanking sequences including the DUX4 pA signal. At the genetic, epigenetic and transcriptional level, D4Z4-12.5 and D4Z4-2.5 mice recapitulate normal and disease alleles, respectively.^{ref} While in D4Z4-12.5 mice, the D4Z4 array is highly methylated and contains high levels of the repressive H3K9me3 modification, in D4Z4-2.5 mice, there is a loss of CpG methylation and H3K9me3 comparable to FSHD alleles (Zeng, et al., 2009; de Greeg, et al., 2009). Consequently D4Z4-12.5 mice can efficiently suppress DUX4 somatic cells, while D4Z4-2.5 mice show a DUX4 expression profile of FSHD patients, including the variegated expression pattern of DUX4 protein in sporadic myonuclei. DUX4 is expressed in D4Z4-2.5 ES cells (like human ES cells) and gets progressively silenced during early development due to an increasing heterochromatic organization with differentiation, similar to human FSHD and control iPS cells (Snider, et al., 2010). Like in humans, DUX4 is highly expressed in the germline of both models (Snider, et al., 2010).

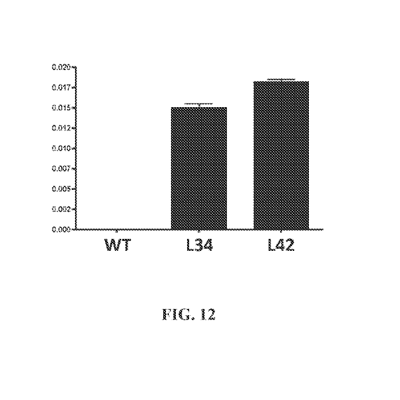

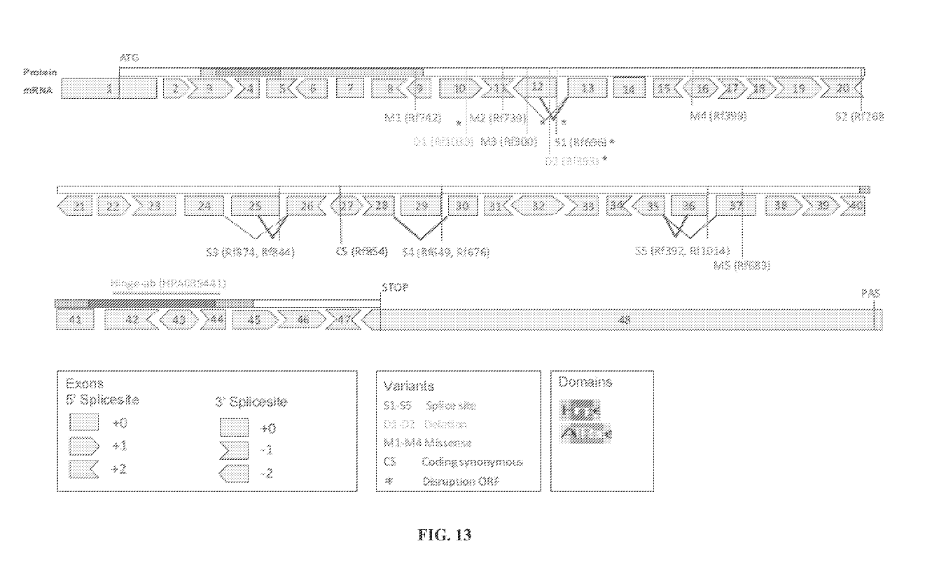

Smchd1 also binds to D4Z4 in D4Z4-12.5 and D4Z4-2.5 mice as evidenced by ChIP analysis (FIG. 12). This demonstrates that the epigenetic regulation of D4Z4 is evolutionary conserved and provides a rationale to study the role of SMCHD1 in the epigenetic regulation of D4Z4 during development and in tissues which are difficult to study in humans and without interference of many other highly homologous loci in the human genome. The preliminary cross of D4Z4-2.5 mice with Smchd^{+/-} mice shows that hapolinsufficiency of Smchd1 severely aggravates the phenotype of D4Z4-2.5 mice. While these mice are viable, at 4 weeks of age they have half the body weight of their littermates. Altogether, studies in mouse show that the epigenetic repression of DUX4 in somatic cells is evolutionary conserved and that the mouse models can be used to study these mechanisms.

### REFERENCES

The following references, to the extent that they provide exemplary procedural or other details supplementary to those set forth herein, are specifically incorporated herein by reference.
U.S. Pat. No. 4,683,202
U.S. Pat. No. 5,288,644
U.S. Pat. No. 5,399,363
U.S. Pat. No. 5,466,468
U.S. Pat. No. 5,466,468
U.S. Pat. No. 5,543,158
U.S. Pat. No. 5,580,579
U.S. Pat. No. 5,629,001
U.S. Pat. No. 5,641,515
U.S. Pat. No. 5,641,515
U.S. Pat. No. 5,725,871
U.S. Pat. No. 5,756,353
U.S. Pat. No. 5,780,045
U.S. Pat. No. 5,792,451
U.S. Pat. No. 5,804,212
U.S. Pat. No. 6,613,308
U.S. Pat. No. 6,753,514
U.S. Patent Publn. 2004/001409
Aartsma-Rus, Mol. Biol., 867:117-129, 2012.
Antibodies: A Laboratory Manual, Cold Spring Harbor Laboratory, Cold Spring Harbor Press, Cold Spring Harbor, N.Y., 1988.
Ausubel et al., In: Current Protocols in Molecular Biology, John Wiley & Sons, NY, 631-636, 2003.
Balog et al., Epigenetics, 7, 2012.
Bamshad et al., Nat. Rev. Genet., 12:745-755, 2011.
Bhalla et al., Mol. Biol. Cell, 13:632-645, 2002.
Blewitt et al., Nat. Genet., 40:663-669, 2008.
Blewitt et al., Proc. Natl. Acad. Sci. USA, 102:7629-7634, 2005.
Bodega et al., BMC. Biol., 7:41, 2009.
Cabianca et al., Cell, 149:819-831, 2012.
Chuang et al., Cell, 79:459-474, 1994.
Church and Gilbert, Proc. Natl. Acad. Sci. USA, 81:1991-1995, 1984.
Clark et al., Nucleic Acids Res. 22:2990-2997, 1994.
Cotton et al., Proc. Natl. Acad. Sci. USA, 85:4397-4401, 1985.
de Greef et al., Hum. Mutat., 30:1449-1459, 2009.
de Greef et al., Neurology, 75:1548-1554, 2010a.
de Greef J C, et al., Neurology. 75 (17):1548-1554, 2010a.
De Jager et al., Semin. Nucl. Med., 23(2):165-179, 1993.
Dej et al., Genetics, 168:895-906, 2004.
Dixit et al., Proc. Natl. Acad. Sci. USA, 104:18157-18162, 2007.
Doolittle and Ben-Zeev, Methods Mol, Biol, 109:215-237, 1999.
Duhl et al., Nat. Genet., 8:59-65, 1994.
Eckert et al., PCR Methods and Applications, 1(1):17-24, 1991.
Flavell et al., Cell, 15:25, 1978.
Frommer et al.: Proc. Natl. Acad. Sci. USA, 89:1827-1831, 1992.
Gabriels et al., Gene, 236:25-32, 1999.
Geever et al., Proc. Natl. Acad. Sci. USA, 78:5081, 1981.
Geier and Modrich, J. Biol. Chem., 254:1408-1413, 1979.
Geng et al., Dev. Cell, 22:38-51, 2012.
Geng et al., Dev. Cell, 22:38-51, 2012.
Guatelli et al., Proc. Nat. Acad. Sci. USA, 87:1874-1878, 1990.
Gulbis and Galand, Hum. Pathol. 24(12), 1271-1285, 1993.
Hewitt et al., Hum. Mol. Genet., 3:1287-1295, 1994.
Hwang et al., Crit. Rev. Ther. Drug Carrier Syst., 15(3):243-284, 1998.
Jiang et al., Hum. Mol. Genet., 12:2909-2921, 2003.
Kanno et al., Nat. Genet., 40:670-675, 2008.
Kwoh et al., Proc. Natl. Acad. Sci. USA, 86:1173, 1989.
Landegren et al., Science, 241:1077-1080, 1988.
Lemmers et al., Am. J. Hum. Genet. 81 (5): 884-894, 2007.
Lemmers et al., Ann. Neurol., 50:816-819, 2001.
Lemmers et al., Nat. Genet., 32:235-236, 2002.
Lemmers et al., Science, 329:1650-1653, 2010a.
Lemmers, et al., Am J Hum Genet. 75:1124-1130, 2004.
Lemmers, et al., Am J Hum Genet. 86 (3):364-377, 2010b.
Lemmers, et al., Nat. Genet. 44(12):1370-1374, 2012.
Lieb et al., Science, 274:1732-1736, 1996.
Lyle et al., Genetics, 28:389-397, 1995.
Marinus and Morris, J. Bacteriol., 114:1143-1150, 1973.
Mathiowitz et al., Nature, 386(6623):410-414, 1997.
Mattila et al., Nucleic Acids Res., 19:4967, 1991.
May and Hattman, J. Bacteriol., 123:768-770, 1975.
McPherson et al., PCR Basics: From Background to Bench, Springer Verlag, 2000.
Myers et al., Science, 230:1242, 1985.
Nakamura et al., In: Handbook of Experimental Immunology (4^{th} Ed.), Weir et al. (Eds.), 1:27, Blackwell Scientific Publ., Oxford, 1987.
Nelson et al., Nat. Protoc., 1:179-185, 2006.
Orita et al., Genomics, 5:874-879, 1989.
PCR Primer: A Laboratory Manual; McPherson et al., 2000;
Pfaffl, Nucleic Acids Res., 29(9):e45, 2001.
Prevalence of rare diseases: Bibliographic data, www.orpha.net, November 2008 Number 1, Orphanet Report Series
Raca et al., Genet. Test., 8(4):387-94, 2004.
Rakyan et al., Trends Genet., 18:348-351, 2002.
Redon et al., Nature, 444:444-454, 2006.
Remington's Pharmaceutical Sciences, 15^{th} Ed., 1035-1038 and 1570-1580, 1990.
Remington's Pharmaceutical Sciences, 18th Ed. Mack Printing Company, 1289-1329, 1990. Sacconi, et al., J Med. Genet. 49 (1):41-46, 2012.
Saiki et al., Nature, 324:163-166, 1986.
Sanger et al., Proc. Natl. Acad. Sci. USA, 74:5463-5467, 1977.
Sheffield et al., Proc. Natl. Acad. Sci. USA, 86:232-236, 1989.
Siegfried and Cedar, Curr. Biol., 7:r305-307, 1997.
Snider et al., Hum. Mol. Genet., 18:2414-2430, 2009.
Snider et al., PLoS. Genet., 6:e1001181, 2010.
Spurlock et al., Muscle Nerve, 42:820-821, 2010.
Statland and Tawil, Curr. Opin. Neurol., 24:423-428, 2011.
Takenaga et al., J. Control Release, 52(1-2):81-87, 1998.
Tawil & van der Maarel, Muscle Nerve. 34:1-15, 2006.
Thomas et al., J. Med. Genet., 44:215-218, 2007.
Underhill et al., Genome Research, 7(10):996-1005, 1997.
van der Maarel et al., Trends Mol. Med., 17:252-258, 2011.
van Deutekom et al., Hum. Mol. Genet., 2:2037-2042, 1993.
van Overveld et al., Ann. Neurol., 58:569-576, 2005.
van Overveld et al., Nat. Genet., 35:315-317, 2003.
Wijmenga et al., Nat. Genet., 2:26-30, 1992.
Wu and Wallace, Genomics, 4:560-569, 1989.
Zeng et al., PLoS. Genet., 5:e1000559, 2009.
Zeschnigk et al., Hum. Mol. Genet., 6:387-395, 1997.

1. An isolated DNA molecule comprising a non-genomic sequence of human SMCHD1 (Structural maintenance of chromosomes flexible hinge domain-containing 1) wherein the sequence comprises a SMCHD1 gene variant that reduces SMCHD1 activity in a cell compared to a cell with a wild-type SMCHD1 sequence.

2. The isolated DNA molecule of claim 1, wherein the SMCHD1 gene variant is a deletion variant, a splice-site variant, or a missense variant.

3. The isolated DNA molecule of any of claims 1-2, wherein the SMCHD1 gene variant is g.2697999_{—}26098003del, g.2700705G>C, g.2700743T>C, g.2700875_{—}2700875del, g.2701019A>G, g.2707565C>T, g.2722661G>A, g.2732488_{—}2732492del, g.2739448T>A, g.2743927G>A, g.2762234G>A, or g.2763729T>C.

4. The isolated DNA molecule of any of claim 1-3, wherein the molecule has fewer than 500 nucleotides.

5. The isolated DNA molecule of any of claims 1-4, wherein the molecule is labeled.

6. The isolated DNA molecule of claim 5, wherein the label is fluorescent, enzymatic, colorimetric, metallic, or radioactive.

7. The isolated DNA molecule of claim 5, wherein the label comprises a detectable compound or substance.

8. An isolated nucleic acid fragment comprising a SMCHD1 gene variant, wherein the presence of the variant in a subject reduces SMCHD1 protein levels in the subject as compared to a subject that does not have the variant.

9. The isolated nucleic acid fragment of claim 8, wherein the variant of SMCHD1 gene comprises a deletion variant, a splice-site variant, or a missense variant.

10. The isolated nucleic acid fragment of any of claims 8-9, wherein the variant of SMCHD1 gene comprises a mutant selected from the group consisting of a deletion mutant of g.2697999_{—}26098003del, a missense mutant of g.2700705G>C, a missense mutant of g.2700743T>C, a deletion mutant of g.2700875_{—}2700875del, a 5′ splice site mutant of g.2701019A>G, a missense mutant of g.2707565C>T, a 5′ splice site mutant of g.2722661G>A, a 5′ splice site mutant of g.2732488_{—}2732492del, a coding synonymous mutant of g.2739448T>A, a 5′ splice mutant of g.2743927G>A, a 5′ splice mutant of g.2762234G>A, and a missence mutant of g.2763729T>C.

11. A method for detecting a mutation associated with facioscapulohumeral dystrophy 2 (FSHD2) comprising assaying for the presence of a variant in one or both alleles of a SMCHD1 (Structural maintenance of chromosomes flexible hinge domain-containing 1) gene in a sample from the subject, wherein the variant is a mutation associated with a reduction of SMCHD1 activity compared to a wildtype SMCHD1 gene.

12. The method of claim 11, further comprising detecting the presence of the variant in one or both alleles in the sample from the subject.

13. The method of claim 11 or 12, wherein the sample is a blood sample.

14. The method of any of claims 11-13, wherein a variant in both alleles of a SMCHD1 gene is detected.

15. The method of any one of claims 11-14, further comprising identifying the subject as having a biomarker indicative of FSHD2.

16. The method of any one of claims 11-15, wherein the variant comprises a deletion variant, a splice-site variant, or a missense variant.

17. The method of any one of claims 11-16, wherein the variant is g.2697999_{—}26098003del, g.2700705G>C, g.2700743T>C, g.2700875_{—}2700875del, g.2701019A>G, g.2707565C>T, g.2722661G>A, g.2732488_{—}2732492del, g.2739448T>A, g.2743927G>A, g.2762234G>A, or g.2763729T>C.

18. The method of any one of claims 11-17, wherein detecting comprises sequencing one or both alleles.

19. The method of any one of claims 11-18, further comprising isolating nucleic acids from the sample.

20. The method of claim 19, wherein genomic DNA is isolated from the sample.

21. The method of claim 19, wherein RNA is isolated from the sample.

22. The method of claim 21, further comprising synthesizing DNA complementary (cDNA) to the isolated RNA.

23. The method of any one of claims 18-19, wherein the sequencing comprises performing genome sequencing, exome sequencing, chain terminating sequencing, restriction digestion, allele-specific polymerase reaction, single-stranded conformational polymorphism analysis, genetic bit analysis, temperature gradient gel electrophoresis, or ligase chain reaction.

24. The method of any one of claims 11-23, further comprising identifying the subject as a being a carrier of an FSHD2 mutation or at risk for FSHD2.

25. A method for detecting facioscapulohumeral dystrophy (FSHD) in a subject comprising assaying for SMCHD1 expression in a sample from the subject and identifying the subject as having a biomarker for FSHD after determining the sample having reduced SMCHD1 expression as compared to a SMCHD1 control or reference.

26. The method of claim 25, wherein SMCHD1 expression is assayed by measuring SMCHD1 mRNA in the sample.

27. The method of claim 25, wherein SMCHD1 expression is assayed by measuring SMCHD1 protein in the sample.

28. The method of claim 24, wherein detecting comprises quantifying mRNA by real time PCR.

29. The method of any one of claims 25-28, wherein the sample is a blood sample.

30. The method of any one of claims 25-29, further comprising diagnosing FSHD in a subject comprising obtaining a sample from the subject, and detecting reduced SMCHD1 mRNA expression, as compared to normal controls.

31. A method for detecting facioscapulohumeral dystrophy (FSHD) in a subject comprising obtaining a sample from the subject, and detecting reduced SMCHD1 protein expression, as compared to normal controls.

32. The method of claim 31, wherein detecting comprises immunologic detection or mass spectrometry.

33. The method of any one of claims 31-32, wherein the sample is a blood sample.

34. The method of any one of claims 31-33, further comprising diagnosing FSHD in a subject comprising obtaining a sample from the subject, and detecting reduced SMCHD1 protein expression, as compared to normal controls.

35. A method for detecting facioscapulohumeral dystrophy (FSHD) in a subject comprising obtaining a sample from the subject, and detecting reduced level of binding between SMCHD1 and D4Z4 array, as compared to normal controls.

36. The method of claim 35, wherein detecting comprising chromatin immunoprecipitation.

37. The method of any one of claims 35-36, wherein the sample is a blood sample.

38. The method of any one of claims 35-37, further comprising diagnosing FSHD in a subject comprising obtaining a sample from the subject, and detecting reduced level of binding between SMCHD1 and D4Z4 array, as compared to normal controls.

39. A method for detecting a variant of SMCHD1 gene in a subject wherein the variant of SMCHD1 gene associates with FSHD2, wherein the presence of the variant of SMCHD1 gene reduces SMCHD1 protein levels in a subject as compared to a subject that does not have the SMCHD1 gene variant.

40. The method of claim 39, wherein the variant of SMCHD1 gene comprises a mutant selected from the group consisting of a deletion mutant of g.2697999_{—}26098003del, a missense mutant of g.2700705G>C, a missense mutant of g.2700743T>C, a deletion mutant of g.2700875_{—}2700875del, a 5′ splice site mutant of g.2701019A>G, a missense mutant of g.2707565C>T, a 5′ splice site mutant of g.2722661G>A, a 5′ splice site mutant of g.2732488_{—}2732492del, a coding synonymous mutant of g.2739448T>A, a 5′ splice mutant of g.2743927G>A, a 5′ splice mutant of g.2762234G>A, and a missence mutant of g.2763729T>C.

41. A method for detecting a variant of SMCHD1 gene in a subject, wherein the subject is at risk of developing FSHD or exhibiting symptoms of FSHD, wherein the presence of the variant of SMCHD1 gene reduces SMCHD1 protein levels in a subject as compared to a subject that does not have the SMCHD1 gene variant.

42. The method of claim 41, wherein the variant of SMCHD1 gene comprises a mutant selected from the group consisting of a deletion mutant of g.2697999_{—}26098003del, a missense mutant of g.2700705G>C, a missense mutant of g.2700743T>C, a deletion mutant of g.2700875_{—}2700875del, a 5′ splice site mutant of g.2701019A>G, a missense mutant of g.2707565C>T, a 5′ splice site mutant of g.2722661G>A, a 5′ splice site mutant of g.2732488_{—}2732492del, a coding synonymous mutant of g.2739448T>A, a 5′ splice mutant of g.2743927G>A, a 5′ splice mutant of g.2762234G>A, and a missence mutant of g.2763729T>C.

43. A method for diagnosing a subject comprising obtaining a sample from a subject; receiving information about the level of SMCHD1 expression in the sample compared to a control or reference level, and diagnosing the subject as having or being at risk for FSHD2 after receiving information that the level of SMCHD1 expression in the sample is reduced compared to the control or reference level.

44. The method of claim 43, further comprising treating the subject for FSHD2.

45. A kit for detecting facioscapulohumeral dystrophy (FSHD) in a subject, wherein the kit comprises an agent for detecting the presence of a variant of SMCHD1 (Structural maintenance of chromosomes flexible hinge domain-containing 1) gene in a sample from the subject, wherein the presence of the variant in a subject reduces SMCHD1 protein levels in the subject as compared to a subject that does not have the variant.

46. The kit of claim 45, wherein the variant of SMCHD1 gene comprises a mutant selected from the group consisting of a deletion mutant of g.2697999_{—}26098003del, a missense mutant of g.2700705G>C, a missense mutant of g.2700743T>C, a deletion mutant of g.2700875_{—}2700875del, a 5′ splice site mutant of g.2701019A>G, a missense mutant of g.2707565C>T, a 5′ splice site mutant of g.2722661G>A, a 5′ splice site mutant of g.2732488_{—}2732492del, a coding synonymous mutant of g.2739448T>A, a 5′ splice mutant of g.2743927G>A, a 5′ splice mutant of g.2762234G>A, and a missence mutant of g.2763729T>C.

47. A kit for detecting facioscapulohumeral dystrophy (FSHD) in a subject, wherein the kit comprises an agent for detecting reduced SMCHD1 mRNA expression in a sample from the subject.

48. A kit for detecting facioscapulohumeral dystrophy (FSHD) in a subject, wherein the kit comprises an agent for detecting reduced SMCHD1 protein expression in a sample from the subject.

49. A kit for detecting facioscapulohumeral dystrophy (FSHD) in a subject, wherein the kit comprises an agent for detecting reduced level of binding between SMCHD1 and D4Z4 arrays in a sample from the subject.


SEQUENCE LISTING (40 sequences)

SEQ ID NO: 1 - protein, 2005 aa, Homo sapiens
MAAADGGGPGGASVGTEEDGGGVGHRTVYLFDRREKESELGDRPLQVGERSDYAGFRACVCQTLGISPEEKFVITTTSRKEITCDNFDETVKDGVTLYLLQSVNQLLLTATKERIDFLPHYDTLVKSGMYEYYASEGQNPLPFALAELIDNSLSATSRNIGVRRIQIKLLFDETQGKPAVAVIDNGRGMTSKQLNNWAVYRLSKFTRQGDFESDHSGYVRPVPVPRSLNSDISYFGVGGKQAVFFVGQSARMISKPADSQDVHELVLSKEDFEKKEKNKEAIYSGYIRNRKPSDSVHITNDDERFLHHLIIEEKEKDSFTAVVITGVQPEHIQYLKNYFHLWTRQLAHIYHYYIHGPKGNEIRTSKEVEPFNNIDIEISMFEKGKVPKIVNLREIQDDMQTLYVNTAADSFEFKAHVEGDGVVEGIIRYHPFLYDRETYPDDPCFPSKLKDEDDEDDCFILEKAARGKRPIFECFWNGRLIPYTSVEDFDWCTPPKKRGLAPIECYNRISGALFTNDKFQVSTNKLTFMDLELKLKDKNTLFTRILNGQEQRMKIDREFALWLKDCHEKYDKQIKFTLFKGVITRPDLPSKKQGPWATYAAIEWDGKIYKAGQLVKTIKTLPLFYGSIVRFFLYGDHDGEVYATGGEVQIAMEPQALYDEVRTVPIAKLDRTVAEKAVKKYVEDEMARLPDRLSVTWPEGDELLPNEVRPAGTPIGALRIEILNKKGEAMQKLPGTSHGGSKKLLVELKVILHSSSGNKEIISHISQHGGKWPYWFKKMENIQKLGNYTLKLQVVLNESNADTYAGRPLPSKAIKFSVKEGKPEKFSFGLLDLPFRVGVPFNIPLEFQDEFGHTSQLVTDIQPVLEASGLSLHYEEITKGPNCVIRGVTAKGPVNSCQGKNYNLKVTLPGLKEDSQILKIRLLPGHPRRLKVKPDSEILVIENGTAFPFQVEVLDESDNITAQPKLIVHCKFSGAPNLPVYVVDCSSSGTSILTGSAIQVQNIKKDQTLKARIEIPSCKDVAPVEKTIKLLPSSHVARLQIFSVEGQKAIQIKHQDEVNWIAGDIMHNLIFQMYDEGEREINITSALAEKIKVNWTPEINKEHLLQGLLPDVQVPTSVKDMRYCQVSFQDDHVSLESAFTVRPLPDEPKHLKCEMKGGKTVQMGQELQGEVVIIITDQYGNQIQAFSPSSLSSLSIAGVGLDSSNLKTTFQENTQSISVRGIKFIPGPPGNKDLCFTWREFSDFIRVQLISGPPAKLLLIDWPELKESIPVINGRDLQNPIIVQLCDQWDNPAPVQHVKISLTKASNLKLMPSNQQHKTDEKGRANLGVFSVFAPRGEHTLQVKAIYNKSIIEGPIIKLMILPDPEKPVRLNVKYDKDASFLAGGLFTDFMISVISEDDSIIKNINPARISMKMWKLSTSGNRPPANAETFSCNKIKDNDKEDGCFYFRDKVIPNKVGTYCIQFGFMMDKTNILNSEQVIVEVLPNQPVKLVPKIKPPTPAVSNVRSVASRTLVRDLHLSITDDYDNHTGIDLVGTIIATIKGSNEEDTDTPLFIGKVRTLEFPFVNGSAEIMSLVLAESSPGRDSTEYFIVFEPRLPLLSRTLEPYILPFMFYNDVKKQQQMAALTKEKDQLSQSIVMYKSLFEASQQLLNEMKCQVEEARLKEAQLRNELKIHNIDIPTTQQVPHIEALLKRKLSEQEELKKKPRRSCTLPNYTKGSGDVLGKIAHLAQIEDDRAAMVISWHLASDMDCVVTLTTDAARRIYDETQGRQQVLPLDSIYKKTLPDWKRSLPHFRNGKLYFKPIGDPVFARDLLTFPDNVEHCETVFGMLLGDTIILDNLDAANHYRKEVVKITHCPTLLTRDGDRIRSNGKFGGLQNKAPPMDKLRGMVFGAPVPKQCLILGEQIDLLQQYRSAVCKLDSVNKDLNSQLEYLRTPDMRKKKQELDEHEKNLKLIEEKLGMTPIRKCNDSLRHSPKVETTDCPVPPKRMRREATRQNRIITKTDV

SEQ ID NO: 2 - DNA, 11 bp, Artificial Sequence
  Synthetic primer
acnnnngtayc

SEQ ID NO: 3 - DNA, 12 bp, Artificial Sequence
  Synthetic primer
cgannnnnntgc

SEQ ID NO: 4 - DNA, 11 bp, Artificial Sequence
  Synthetic primer
acnnnnnctcc

SEQ ID NO: 5 - DNA, 12 bp, Artificial Sequence
  Synthetic primer
caannnnngtgg

SEQ ID NO: 6 - DNA, 29 bp, Artificial Sequence
  Synthetic primer
cgtaactataacggtcctaaggtagcgaa

SEQ ID NO: 7 - DNA, 18 bp, Artificial Sequence
  Synthetic primer
tagggataacagggtaat

SEQ ID NO: 8 - DNA, 30 bp, Artificial Sequence
  Synthetic primer
tggcaaacagctattatgggtattatgggt

SEQ ID NO: 9 - DNA, 30 bp, Artificial Sequence
  Synthetic primer
atctatgtcgggtgcggagaaagaggtaat

SEQ ID NO: 10 - DNA, 20 bp, Artificial Sequence
  Synthetic primer
atttccacctttggacacga

SEQ ID NO: 11 - DNA, 20 bp, Artificial Sequence
  Synthetic primer
tgctgacctggaatttgtca

SEQ ID NO: 12 - DNA, 21 bp, Artificial Sequence
  Synthetic primer
tcccctcttttatggaagcat

SEQ ID NO: 13 - DNA, 20 bp, Artificial Sequence
  Synthetic primer
ttcctggaagcttttgcatt

SEQ ID NO: 14 - DNA, 20 bp, Artificial Sequence
  Synthetic primer
catggaggaaaatggcctta

SEQ ID NO: 15 - DNA, 20 bp, Artificial Sequence
  Synthetic primer
attcagccagttcttgaagc

SEQ ID NO: 16 - DNA, 22 bp, Artificial Sequence
  Synthetic primer
tgccttgacaagagtttacagg

SEQ ID NO: 17 - DNA, 24 bp, Artificial Sequence
  Synthetic primer
tctggaaccagtattttaacagga

SEQ ID NO: 18 - DNA, 21 bp, Artificial Sequence
  Synthetic primer
ggatagcgggtgatattatgc

SEQ ID NO: 19 - DNA, 20 bp, Artificial Sequence
  Synthetic primer
ttgcacatcaggaagcagac

SEQ ID NO: 20 - DNA, 20 bp, Artificial Sequence
  Synthetic primer
ctggggttggacttgatagc

SEQ ID NO: 21 - DNA, 20 bp, Artificial Sequence
  Synthetic primer
aaccccagcaattgacaaag

SEQ ID NO: 22 - DNA, 20 bp, Artificial Sequence
  Synthetic primer
ggtgctggattatcccactg

SEQ ID NO: 23 - DNA, 20 bp, Artificial Sequence
  Synthetic primer
ctgttggtttgaaggcatga

SEQ ID NO: 24 - DNA, 22 bp, Artificial Sequence
  Synthetic primer
tccagtttggttttatgatgga

SEQ ID NO: 25 - DNA, 20 bp, Artificial Sequence
  Synthetic primer
ttcacgaaggggaattcaag

SEQ ID NO: 26 - DNA, 20 bp, Artificial Sequence
  Synthetic primer
taagtgctgccatttgttgc

SEQ ID NO: 27 - DNA, 21 bp, Artificial Sequence
  Synthetic primer
cgacagattgtccagttcctc

SEQ ID NO: 28 - DNA, 20 bp, Artificial Sequence
  Synthetic primer
ccaatggcctcttctctctg

SEQ ID NO: 29 - DNA, 21 bp, Artificial Sequence
  Synthetic primer
tcctaagaagagagggcttgc

SEQ ID NO: 30 - DNA, 26 bp, Artificial Sequence
  Synthetic primer
ttaatctcaggagtccaattaacttt

SEQ ID NO: 31 - DNA, 20 bp, Artificial Sequence
  Synthetic primer
tccaggtcctcctggaaata

SEQ ID NO: 32 - DNA, 19 bp, Artificial Sequence
  Synthetic primer
tccagcaccggtacaacat

SEQ ID NO: 33 - DNA, 21 bp, Artificial Sequence
  Synthetic primer
cctgcctaatcaacctgtgaa

SEQ ID NO: 34 - DNA, 20 bp, Artificial Sequence
  Synthetic primer
tactggcaactgagcgaaca

SEQ ID NO: 35 - DNA, 20 bp, Artificial Sequence
  Synthetic primer
cccattggagatccagtctt

SEQ ID NO: 36 - DNA, 20 bp, Artificial Sequence
  Synthetic primer
ccgcatccagattatccaaa

SEQ ID NO: 37 - DNA, 20 bp, Artificial Sequence
  Synthetic primer
tggataaacttcggggaatg

SEQ ID NO: 38 - RNA, 21 nt, Artificial Sequence
  Synthetic primer
guccagaaauuaguugcacuc

SEQ ID NO: 39 - RNA, 20 nt, Artificial Sequence
  Synthetic primer
gauuaggcaggacuucaacu

SEQ ID NO: 40 - RNA, 15 nt, Artificial Sequence
  Synthetic primer
ggcugcuuugcccuc